(19) United States
(12) Patent Application Publication
(10) Pub. No.: US 2004/0029864 A1
(43) Pub. Date: Feb. 12, 2004

(54) Treatment of colds and cough with a combination of a cyclooxygenase-2 selective inhibitor and a colds and cough active ingredient and compositions thereof

(75) Inventor: Stephen P. MacMillan, Newtown, PA (US)

Correspondence Address: Charles E. Dunlap, Keenan Building, Third Floor, 1330 Lady Street, Columbia, SC, 29201, US

(73) Assignee: Pharmacia Corporation, St. Louis, MO

(21) Appl. No.: 10/357,747

(22) Filed: Feb. 4, 2003

Related U.S. Application Data
(60) Provisional application No. 60/354,135, filed on Feb. 4, 2002.

(51) Int. Cl. A61K 31/55; A61K 31/415; A61K 31/495

(52) U.S. Cl. 514/217.05; 514/255.04; 514/406

(57) ABSTRACT

A method for the treatment, prevention and amelioration of colds and/or cough in a subject in need of such treatment, prevention and amelioration, comprises administering to the subject a cyclooxygenase-2 selective inhibitor or prodrug thereof and one or more colds and cough active ingredient. Compositions, pharmaceutical compositions and kits for practicing the method are also disclosed.

### CROSS-REFERENCE TO RELATED PATENTS AND PATENT APPLICATIONS

The subject matter of the present invention is related to and claims the benefit of co-pending and commonly assigned U.S. Provisional Patent Application Serial No. 60/354,135, filed on Feb. 4, 2002, which application is hereby incorporated herein by reference in its entirety.

### BACKGROUND OF THE INVENTION

(1) Field of the Invention

The present invention relates to the treatment of colds and coughs, and more particularly to the treatment of colds and coughs by administering to a subject a combination of a cyclooxygenase-2 selective inhibitor and a colds and cough active ingredient.

(2) Description of the Related Art

The common cold is an acute viral infection of the mucous membranes of the nose and throat, often involving the sinuses. The typical sore throat, sneezing, and fatigue can be accompanied by body aches, headache, low fever, and chills. The congested and discharging mucous membrane may become a fertile ground for s secondary bacterial infection that can spread to the larynx, bronchi, lungs, or ears. Uncomplicated infections usually last from three to ten days.

Colds are caused by any one of up to 200 viruses—such as the rhinoviruses, coronaviruses, or respiratory syncytial virus. Infection with a viral strain confers only a temporary immunity to that strain. Colds in infants and young children caused by the respiratory syncytial virus can progress to pneumonia and other complications, and can result in death.

It is believed that there is no treatment for the common cold other than that aimed at relieving symptoms and keeping the body well rested, fed, and hydrated. However, many compounds have been found that are effective in the relief of aches and pain (analgesics—usually non-steroidal anti-inflammatory drugs, or NSAID's), in reducing sneezing and runny nose (antihistamines), for the suppression of coughs (antitussives), for the breakup of nasal and sinus congestion (decongestants), and for helping clear the lungs of excess mucus (expectorants). Many of these medications are available commercially, and more are now being developed.

One promising area for colds medications is the development of new antiviral agents. Older antiviral compounds such as aciclovir have proven to be effective against herpesviruses, and new materials such as dipyridamole, impulsin, and pleconaril have shown promise for the prevention and/or amelioration of colds. See, e.g., Jefferson, T. O. et al., Cochrane Database Syst. Rev., 1:3 CD002743 (2001); and Romero, J. R., Expert. Opin. Investig. Drugs, 10(2):369-379 (2001).

Recently, significant progress has also been made in the field of inflammation, and the development of drugs that show promise for the treatment of the inflammation-related disorders of osteoarthritis and rheumatoid arthritis. It has been known for some time that many of the common non-steroidal antiinflammatory drugs (NSAIDs) modulate prostagiandin synthesis by inhibition of cyclooxygenases that catalyze the transformation of arachidonic acid—the first step in the prostaglandin synthesis pathway. However, the use of high doses of many common NSAIDs can produce severe side effects that limit their therapeutic potential.

In an effort to reduce the unwanted side effects of common NSAIDS, it was discovered that two cyclooxygenases are involved in the transformation of arachidonic acid as the first step in the prostaglandin synthesis pathway. These enzymes have been termed cyclooxygenase-1 (Cox-1) and cyclooxygenase-2 (Cox-2). See, Needleman, P. et al., J. Rheumatol., 24, Suppl.49:6-8 (1997). See, Fu, J. Y., et al., J. Biol. Chem., 265(28):16737-40 (1990).

Cox-1 has been shown to be a constitutively produced enzyme that is involved in many of the non-inflammatory regulatory functions associated with prostaglandins. Cox-2, on the other hand, is an inducible enzyme having significant involvement in the inflammatory process. Inflammation causes the induction of Cox-2, leading to the release of prostanoids, which sensitize peripheral nociceptor terminals and produce localized pain hypersensitivity. See, e.g., Samad, T. A. et al., Nature, 410(6827):471-5 (2001). Many of the common NSAIDs are now known to be inhibitors of both Cox-1 and Cox-2. Accordingly, when administered in sufficiently high levels, these NSAIDs affect not only the inflammatory consequences of Cox-2 activity, but also the beneficial activities of Cox-1.

Recently, compounds that selectively inhibit Cox-2 to a greater extent than the activity of Cox-1 have been discovered. The new Cox-2-selective inhibitors are believed to offer advantages that include the capacity to prevent or reduce inflammation while avoiding harmful side effects associated with the inhibition of Cox-1.

Interestingly, for purposes of the present invention, it has been reported that isolated alkali metal and alkali-earth metal salts of acetaminophen could be used for treatment of mammals in need of an analgesic or antipyretic agent. U.S. Pat. No. 6,160,020 to Ohannesian et al. However, the purpose of the invention was to provide metal salts of acetaminophen with improved aqueous solubility and taste. The acetaminophen salts could be combined with other active ingredients such as analgesics, decongestants, expectorants, antitussives, antihistamines, diuretics, gastrointestinal agents, bronchodilators, and sleep-inducing agents. The analgesic could be supplied by acetylsalicylic acid (aspirin), indomethacin, and Cox-2 inhibitors such as flosulide, nimesulide, celecoxib, 5-(4-aminosu lfonyl-3-fluorophenyl)-4-cyclohexyl-2-methyloxazole, meloxicam, nambumethone, and etodolac, among other compounds. However, no indication was provided that the analgesic should be a Cox-2 selective inhibitor. Furthermore, the additional chemical reactions and separations necessary to provide isolated metal salts of acetaminophen, rather than the acid form of acetaminophen, result in additional expense and require more complex production techniques.

U.S. Pat. Nos. 6,271,253; 6,034,256; 6,077,850; and 6,271,253 to Carter et al. describe the use of certain substituted benzopyran Cox-2 inhibitors for the treatment of inflammation. It is also stated that the substituted benzopyran Cox-2 inhibitors can be used in addition to other anti-inflammatories, and in combination with opioids and other analgesics, codeine, hydrocodone, antihistamines, decongestants, diuretics and antitussive agents.

U.S. Pat. No. 6,303,628 to Nakao et al. describes certain bicycliccarbonyl indole compounds as having Cox-2 selective inhibitory activity, and states that these compounds are useful for treating Cox-2 mediated diseases—including co-administration with such other ingredients as another pain reliever, a potentiator, a decongestant, an antitussive, a prostaglandin, a diuretic, an antihistamine, anticancer agents, and the like.

From the foregoing, it can be seen that a need exists for improved treatment methods and compositions for colds and coughs. It would also be useful if such improved methods and compositions could be provided that combined the effectiveness of Cox-2 selective inhibitors with the effectiveness of one or more compounds that are useful for ameliorating the symptoms of colds and/or cough. Moreover, it would be useful if such methods and compositions avoided the requirement for special forms of active ingredients, in particular, if they could be free of such materials as isolated metal salts of an active ingredient—isolated metal salts of acetaminophen as an example.

### SUMMARY OF THE INVENTION

Briefly, therefore the present invention is directed to a novel method for the treatment, prevention and amelioration of colds and/or cough in a subject in need of such treatment, prevention and amelioration, the method comprising administering to the subject a cyclooxygenase-2 selective inhibitor or prodrug thereof and one or more colds and cough active ingredient.

The present invention is also directed to a novel composition for the treatment, prevention and amelioration of colds and/or cough in a subject in need of such treatment, prevention and amelioration, the composition comprising a cyclooxygenase-2 selective inhibitor and a colds and cough active ingredient.

The present invention is also directed to a novel composition for the treatment, prevention and amelioration of colds and/or cough in a subject in need of such treatment, prevention and amelioration, the composition comprising a cyclooxygenase-2 selective inhibitor selected from the group consisting of celecoxib, parecoxib and valdecoxib, and a colds and cough active ingredient selected from the group consisting of chlorpheniramine, cetirzine, loratadine, codeine, hydrocodone, carbetapentane, dextromethorphan, aspirin, guaifenesin, ephedrine, ephinephrine, phenylephrine, phenylpropanolamine, pseudoephedrine, impulsin, pleconaril, aciclovir, and ganciclovir.

The present invention is also directed to a novel composition for the treatment, prevention and amelioration of colds and/or cough in a subject in need of such treatment, prevention and amelioration, the composition comprising a cyclooxygenase-2 selective inhibitor and a combination of two or more colds and cough active ingredients.

The present invention is also directed to a novel pharmaceutical composition for the treatment, prevention and amelioration of colds and/or cough in a subject in need of such treatment, prevention and amelioration, the composition comprising a cyclooxygenase-2 selective inhibitor, a colds and cough active ingredient, and a pharmaceutically-acceptable excipient.

The present invention is also directed to a novel kit that is suitable for use in the treatment, prevention or amelioration of colds and/or cough, the kit comprises a first dosage form comprising a colds and cough active ingredient and a second dosage form comprising a cyclooxygenase-2 selective inhibitor or prodrug thereof, in quantities which comprise a therapeutically effective amount of the combination of the compounds for the treatment, prevention, or amelioration of colds and/or cough.

Among the several advantages found to be achieved by the present invention, therefore, may be noted the provision of improved treatment methods and compositions for colds and coughs, the provision of such improved methods and compositions that combined the effectiveness of Cox-2 selective inhibitors with the effectiveness of one or more compounds that are useful for ameliorating the symptoms of colds and/or cough, the provision of such methods and compositions that avoided the requirement for special forms of active ingredients, the provision of such methods and compositions that were free of such materials as isolated metal salts of an active ingredient, and in particular, isolated metal salts of acetaminophen.

### DETAILED DESCRIPTION OF THE PREFERRED EMBODIMENTS

In accordance with the present invention, it has been discovered that some or all of the symptoms of colds and cough can be treated, prevented or ameliorated in a subject in need of such treatment, prevention or amelioration by administering to the subject a cyclooxygenase-2 selective inhibitor or prodrug thereof and one or more colds and cough active ingredient. In order to reduce the costs and complications of producing the novel combinations, it has been found that combinations comprising acetaminophen are not required to contain the isolated metal salt of acetaminophen. Indeed, in combinations comprising analgesics, it has been found that it is not required that the analgesic be an isolated metal salt of the analgesic.

In certain embodiments, the compositions of the invention comprise one or more Cox-2 selective inhibitors in combination with two or more colds and cough active ingredients.

In each of the embodiments of the subject methods and compositions, it has been found that the anti-inflammatory and analgesic effects of a Cox-2 selective inhibitor can be enjoyed without the adverse side effects of some other common NSAIDs. Moreover, the novel methods and compositions provide the benefits of the colds and cough active ingredients that are included.

One component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

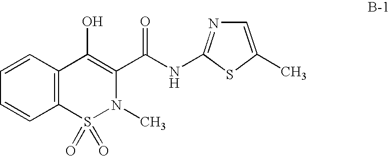

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

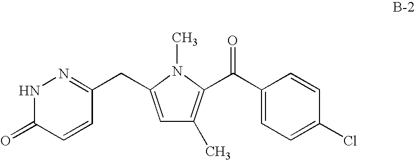

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 1, including the diastereomers, enantiomers, racemates, tautomers, salts, esters, amides and prodrugs thereof.

Benzopyrans that can serve as a cyclooxygenase-2 selective inhibitor of the present invention include substituted benzopyran derivatives that are described in U.S. Pat. No. 6,271,253. One such class of compounds is defined by the general formula shown below in formulas I:

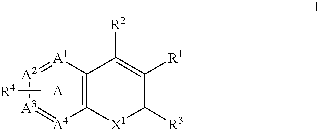

wherein X¹ is selected from O, S, CR^{c} R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b} R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl;

wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes a compound having the structure of formula II:

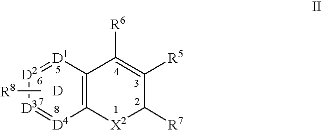

wherein X² is selected from O, S, CR^{c} R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{c} R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl; wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

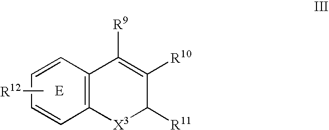

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl;

wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as cyclooxygenase-2 selective inhibitors in the present invention is described by Formulas IV and V:

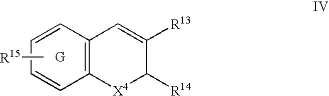

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical; or an isomer or pharmaceutically acceptable salt thereof.

Formula V is:

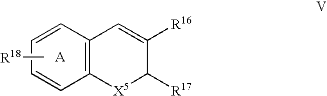

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is carboxyl; R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, d ifluoroethyl, d ifluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or

wherein R² together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The cyclooxygenase-2 selective inhibitor of the present invention can also be a compound having the structure of Formula VI:

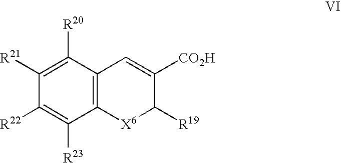

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido, chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective Inhibitors

| Compound | |
|---|---|
| Number | Structural Formula |
| B-3 | 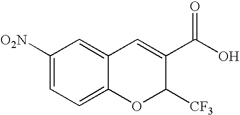 |
| | 6-Nitro-2-trifluoromethyl-2H-1- |
| | benzopyran-3-carboxylic acid |
| B-4 | 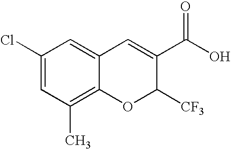 |
| | 6-Chloro-8-methyl-2-trifluoromethyl- |
| | 2H-1-benzopyran-3-Carboxylic acid |
| B-5 | 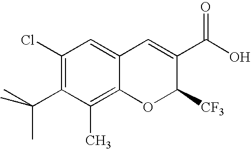 |
| | ((S)-6-Chloro-7-(1,1-dimethylethyl)-2- |
| | (trifluoromethyl-2H-1-benzopyran-3-Carboxylic acid |
| B-6 | 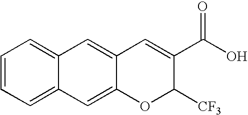 |
| | 2-Trifluoromethyl-2H-naphtho[2,3-b]pyran- |
| | 3-carboxylic acid |
| B-7 | 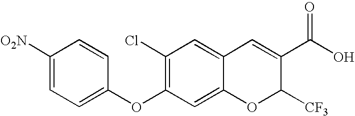 |
| | 6-Chloro-7-(4-nitrophenoxy)-2-(trifluoromethyl)-2H-1- |
| | benzopyran-3-carboxylic acid |
| B-8 | 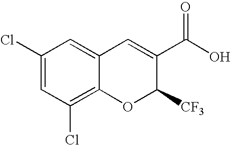 |
| | ((S)-6,8-Dichloro-2-(trifluoromethyl)- |
| | 2H-1-benzopyran-3-carboxylic acid |
| B-9 | 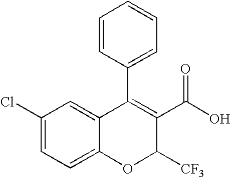 |
| | 6-Chloro-2-(trifluoromethyl)-4-phenyl-2H- |
| | 1-benzopyran-3-carboxylic acid |
| B-10 | 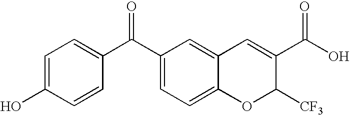 |
| | 6-(4-Hydroxybenzoyl)-2-(trifluoromethyl)- |
| | 2H-1-benzopyran-3-carboxylic acid |
| B-11 | 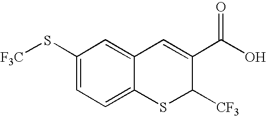 |
| | 2-(Trifluoromethyl)-6-[(trifluoromethyl)thio]- |
| | 2H-1-benzothiopyran-3-carboxylic acid |
| B-12 | 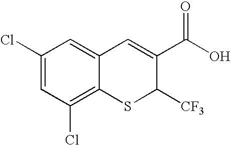 |
| | 6,8-Dichloro-2-trifluoromethyl-2H-1- |
| | benzothiopyran-3-carboxylic acid |
| B-13 | 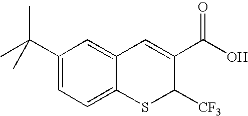 |
| | 6-(1,1-Dimethylethyl)-2-(trifluoromethyl)- |
| | 2H-1-benzothiopyran-3-carboxylic acid |
| B-14 | 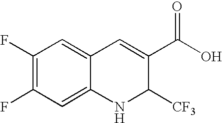 |
| | 6,7-Difluoro-1,2-dihydro-2-(trifluoromethyl)- |
| | 3-quinolinecarboxylic acid |
| B-15 | 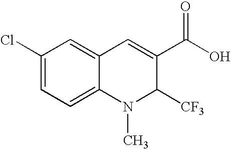 |
| | 6-Chloro-1,2-dihydro-1-methyl-2-(trifluoromethyl)- |
| | 3-quinolinecarboxylic acid |
| B-16 | 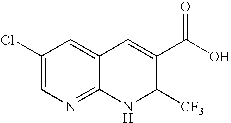 |
| | 6-Chloro-2-(trifluoromethyl)-1,2- |
| | dihydro[1,8]naphthyridine-3-carboxylic acid |
| B-17 | 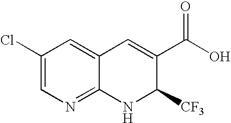 |
| | ((S)-6-Chloro-1,2-dihydro-2-(trifluoromethyl)- |
| | 3-quinolinecarboxylic acid |

Examples of specific compounds that are useful for the cyclooxygenase-2 selective inhibitor include (without limitation):

a1) 8-acetyl-3-(4-fluorophenyl)-2-(4-methylsulfonyl)phenyl-imidazo(1,2-a)pyridine;

a2) 5, 5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-d diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d 9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e 1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-d ien-5-yl]benzenesulfonamide;

e 10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-th iophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i 10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-d ifluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n 10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H— 1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H— 1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H— 1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H— 1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q 10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide; or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of formula VII:

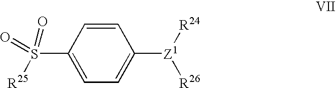

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula VII is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-18 | 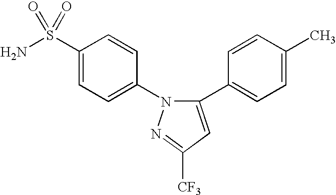 |
| B-19 | 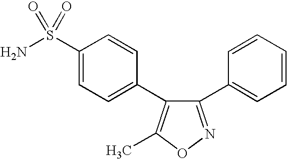 |
| B-20 | 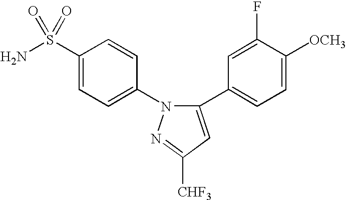 |
| B-21 | 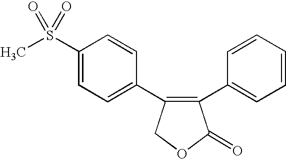 |
| B-22 | 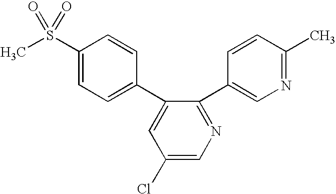 |
| B-23 | 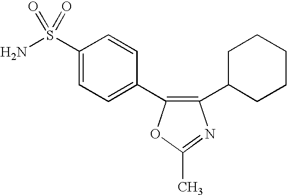 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-1 9, (See, e.g., U.S. Pat. No. 5,633,272), may be advantageously employed as a source of a cyclooxygenase inhibitor.

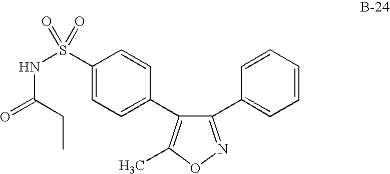

A preferred form of parecoxib is sodium parecoxib.

In another embodiment of the invention, the compound ABT-963 having the formula B-25 that has been previously described in International Publication number WO 00/24719, is another tricyclic cyclooxygenase-2 selective inhibitor which may be advantageously employed.

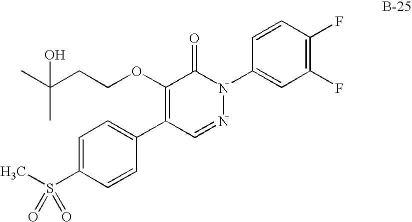

In a further embodiment of the invention, the cyclooxygenase inhibitor can be selected from the class of phenylacetic acid derivative cyclooxygenase-2 selective inhibitors represented by the general structure of Formula VIII:

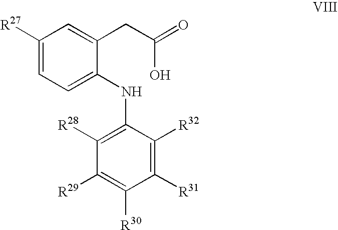

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

A phenylacetic acid derivative cyclooxygenase-2 selective inhibitor that is described in WO 99/11605 is a compound that has the structure shown in Formula VIII,

wherein:

R²⁷ is ethyl;

R²⁸ and R³⁰ are chloro;

R²⁹ and R³¹ are hydrogen; and

R³² is methyl.

Another phenylacetic acid derivative cyclooxygenase-2 selective inhibitor is a compound that has the structure shown in Formula VIII,

wherein:

R²⁷ is propyl;

R²⁸ and R³⁰ are chloro;

R²⁹ and R³¹ are methyl; and

R³² is ethyl.

Another phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula VIII,

wherein:

R²⁷ is methyl;

R²⁸ is fluoro;

R³² is chloro; and

R²⁹ R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other cyclooxygenase-2 selective inhibitors that can be used in the present invention have the general structure shown in formula IX, where the J group is a carbocycle or a heterocycle. Preferred embodiments have the structure:

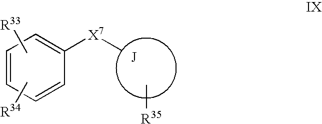

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6-NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N^{-SO}₂CH₃ Na⁺, (L-745337); and

X is S; J is thiophen-2-yl; R³³ is 4-F; there is no R³⁴ group; and R³⁵ is 5-NHSO₂CH₃, (RWJ-63556); and

X is 0; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (06/06/2001); and Iwata, K. et al., in Jpn. J. Pharmacol., 75(2):191-194 (1997).

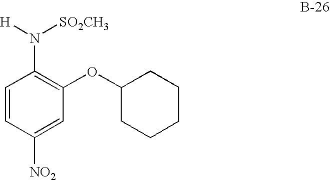

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651. Such diarylmethylidenefuran derivatives have the general formula shown below in formula X:

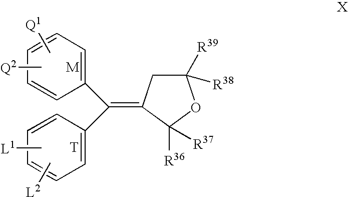

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q¹, Q², L¹ or L² is:

an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R³⁶, R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R³⁶, R³⁷ or R³⁸, R³⁹ are an oxygen atom, or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, 10/04/2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covanlently attached to one or more linkers, as described in U.S. Pat. No. 6,395,724.

Compounds that may act as cyclooxygenase-2 inhibitors include conjugated linoleic acid that is described in U.S. Pat. No. 6,077,868.

Materials that can serve as a cyclooxygenase-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

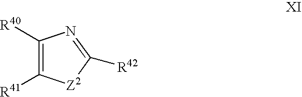

wherein:

Z² is an oxygen atom;

one of R⁴⁰ and R⁴¹ is a group of the formula

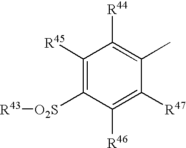

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl, and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

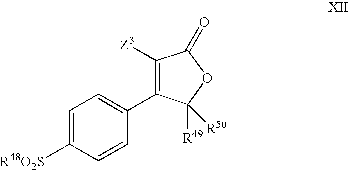

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7) —CO₂H;

R⁴⁸ is selected from the group consisting of NH₂ and CH₃,

R⁴⁹ is selected from the group consisting of:

C₁₋₆ alkyl unsubstituted or substituted with C₃₋₆ cycloalkyl, and C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as cyclooxygenase-2 selective inhibitors include pyridines that are described in U.S. Pat. Nos. 6,369,275, 6,127,545, 6,130,334, 6,204,387, 6,071,936, 6,001,843 and 6,040,450, and which have the general formula described by formula XIII:

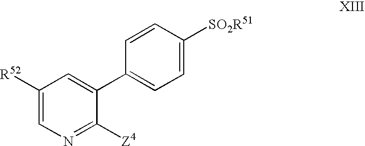

wherein:

R⁵′ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof,

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₁₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j) —C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵, R⁵⁶, R⁵⁷, R⁵⁸ R⁵⁹ R⁶⁰ R⁶¹, R⁶², R⁶³, are each independently chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the cyclooxygenase-2 selective inhibitor of the present invention include diarylbenzopyran derivatives that are described in U.S. Pat. No. 6,340,694. Such diarylbenzopyran derivatives have the general formula shown below in formula XIV:

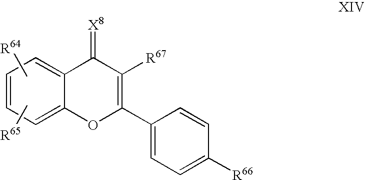

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)NR⁶⁸ wherein n is an integer of 0-2, R⁶⁸ is a hydrogen atom, a C₁-C₆ lower alkyl group, or a group of a formula: NR⁶⁹ R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

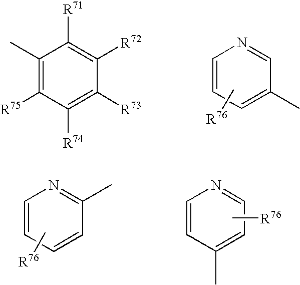

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)NR⁶⁸, a group of a formula: NR⁶⁹ R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the cyclooxygenase-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

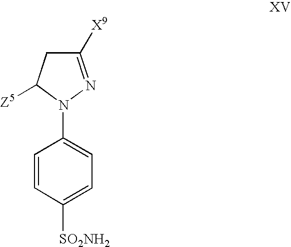

wherein:

X⁹ is selected from the group consisting of C₁-C₆ trihalomethyl, preferably trifluoromethyl; C₁-C₆ alkyl; and an optionally substituted or di-substituted phenyl group of formula XVI:

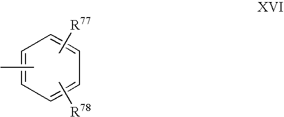

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the cyclooxygenase-2 selective inhibitor of the present invention include heterocycles that are described in U.S. Pat. No. 6,153,787. Such heterocycles have the general formulas shown below in formulas XVII and XVIII:

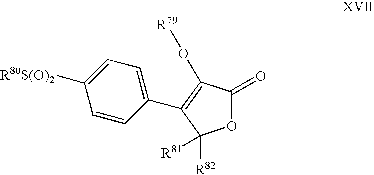

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e)=O,

(f) dioxolane,

(g) CN; and

R⁸⁰ is selected from the group consisting of:

(a) CH₃,

(b) NH₂,

(c) NHC(O)CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

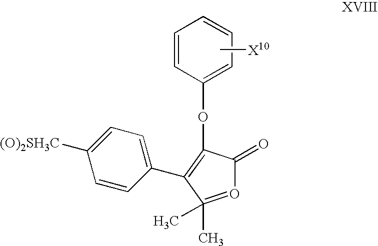

X¹⁰ is fluoro or chloro.

Materials that can serve as the cyclooxygenase-2 selective inhibitor of the present invention include 2,3,5-trisubstituted pyridines that are described in U.S. Pat. No. 6,046,217. Such pyridines have the general formula shown below in formula XIX:

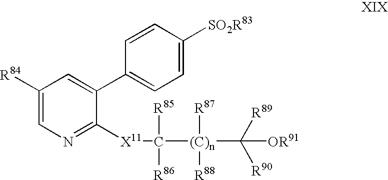

or a pharmaceutically acceptable salt thereof,

wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂ R⁹²,

(i) hydroxy,

(j) —C(R⁹³)(R⁹⁴)—OH,

(k) —C₁₋₆ alkyl-CO₂-R⁹⁵,

(l) C₁₋₆ fluoroalkoxy,

(m) NO₂,

(n) NR⁹⁶ R⁹⁷,

(o) NHCOR⁹⁸;

R⁸⁵ to R⁹⁸ are independantly chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is 0.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the cyclooxygenase-2 selective inhibitor of the present invention include diaryl bicyclic heterocycles that are described in U.S. Pat. No. 6,329,421. Such diaryl bicyclic heterocycles have the general formula shown below in formula XX:

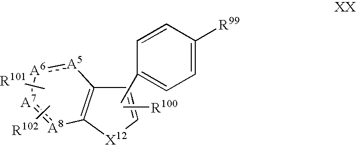

and pharmaceutically acceptable salts thereof wherein:

-A⁵=A⁶_A⁷=A⁸- is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂—CH₂,

(d) —CH₂—CH₂—O—C(O)—, CH₂—O—C(O)—CH₂—O—C(O)—CH₂—CH₂—,

(e) —CH₂—CH₂—C(O)—O—, —CH₂—C(O)—OCH₂—C(O)—O—CH₂—CH₂—,

(f) —C(R¹⁰⁵)₂—O—C(O)—, —C(O)—O—C(R¹⁰⁵)₂—, —C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of:

(a) S(O)₂ CH₃,

(b) S(O)₂ NH₂,

(c) S(O)₂ NHCOCF₃,

(d) S(O)(NH)CH₃,

(e) S(O)(NH)NH₂,

(f) S(O)(NH)NHCOCF₃,

(g) P(O)(CH₃)OH, and

(h) P(O)(CH₃)NH₂;

R¹⁰⁰ is selected from the group consisting of:

(a) C₁₋₆ alkyl,

(b) C₃₋₇, cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10)—CO₂—C₁₋₄ alkyl,

(11) —C(R¹⁰³)(R¹⁰⁴)—OH,

(12) —C(R¹⁰³)(R¹⁰⁴)—O—C₁₋₄ alkyl, and

(13)—C₁₋₆ alkyl-CO₂-R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁₋₄ alkyl;

(e) benzoheteroaryl which includes the benzo fused analogs of (d);

R¹⁰¹ and R¹⁰² are the substituents residing on any position of -A⁵=A⁶-A⁷=A⁸- and are selected independently from the group consisting of:

(a) hydrogen,

(b) CF₃,

(c) CN,

(d) C₁₋₆ alkyl,

(e) Q3 wherein Q3 is Q⁴, CO₂H, C(R¹⁰³)(R¹⁰⁴)OH,

(f) —O-Q⁴,

(g) —S-Q⁴, and

(h) optionally substituted:

(1) —C₁₋₅ alkyl-Q³,

(2) —O—C₁₋₅ alkyl-Q³,

(3) —S—C₁₋₅ alkyl-Q³,

(4) —C-3 alkyl-O—C₁₋₃ alkyl-Q³,

(5) —C₁₋₃ alkyl-S—C₁₋₃ alkyl-Q³,

(6) —C₁₋₅ alkyl-O-Q⁴,

(7) —C₁₋₅ alkyl-S-Q⁴,

wherein the substituent resides on the alkyl chain and the substituent is C₁₋₃ alkyl, and Q³ is Q4, CO₂H, C(R¹⁰³)(R¹⁰⁴)OH Q⁴ is CO₂—C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl;

X⁷ is O, S, NR¹⁰⁷, CO, C(R¹⁰⁷)₂, C(R¹⁰⁷)(OH), —C(R⁰⁷)═C(R⁰⁷)—; —C(R¹⁰⁷)═N—; —N═C(R¹⁰⁷)-.

Compounds that may act as cyclooxygenase-2 inhibitors include salts of 5-amino or a substituted amino 1,2,3-triazole compound that are described in U.S. Pat. No. 6,239,137. The salts are of a class of compounds of formula XXI:

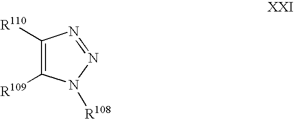

wherein:

R¹⁰⁸ is:

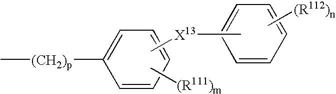

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, loweralkyl, hydroxy, loweralkoxy, amino, loweralkylamino, diloweralkylamino or cyano; and, R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl; R¹⁰⁹ is amino, mono or diloweralkyl amino, acetamido, acetimido, ureido, formamido, formamido or guanidino; and R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Materials that can serve as a cyclooxygenase-2 selective inhibitor of the present invention include pyrazole derivatives that are described in U.S. Pat. No. 6,136,831. Such pyrazole derivatives have the formula shown below in formula XXII:

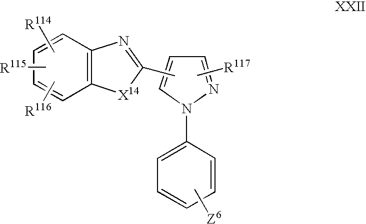

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl; or a pharmaceutically acceptable salt thereof.

Materials that can serve as a cyclooxygenase-2 selective inhibitor of the present invention include substituted derivatives of benzosulphonamides that are described in U.S. Pat. No. 6,297,282. Such benzosulphonamide derivatives have the formula shown below in formula XXIII:

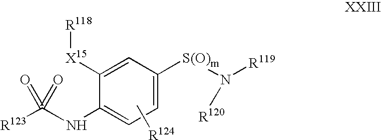

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N— atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;

X¹⁶ denotes halogen, NO₂, —OR¹²¹, —COR¹²¹, —CO₂ R¹²¹, —OCO₂ R¹²¹, —CN, —CONR¹²¹ OR¹²²-CONR²¹ R¹²², —SR²¹, —S(O)R¹²¹, —S(O)₂ R¹²¹, —NR¹²¹ R¹²², —NHC(O)R¹²¹, —NHS(O)₂ R¹²¹;

n denotes a whole number from 0 to 6;

R¹²³ denotes a straight-chained or branched alkyl group with 1-10 C— atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C— atoms, which can optionally be mono- or polysubstituted by halogen, NO₂, —OR¹²¹, COR¹², —CO₂ R 121 OCO₂ R¹²¹, —CN, —CONR¹²¹ OR¹²², —CONR¹² R¹, —SR¹²¹, —S(O)R, —S(O)₂ R¹²¹1-NR¹²¹ R¹²²₇—NHC(O)R¹²¹, NHS(O)₂ R¹²¹, or a polyfluoroalkyl group;

R¹²¹ and R²², independently from one another, denote hydrogen, alkyl, aralkyl or aryl; and

m denotes a whole number from 0 to 2;

and the pharmaceutically-acceptable salts thereof.

Materials that can serve as a cyclooxygenase-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

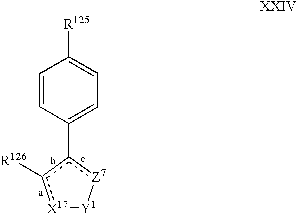

or pharmaceutically acceptable salts thereof wherein:

X¹⁷—Y¹-Z⁷-is selected from the group consisting of:

(a) —CH₂ CH₂ CH₂—,

(b) —C(O)CH₂ CH₂—,

(c) —CH₂ CH₂ C(O)—,

(d) —CR¹²⁹ (R¹²⁹′)—O—C(O)—,

(e) —C(O)—O—CR¹²⁹ (R^{129′})—,

(f) —CH₂—NR¹²⁷-CH₂—,

(g) —CR¹²⁹ (R¹²⁹′)—NR¹²⁷—C(O)—,

(h) —CR¹²⁸=CR^{128′}—S—

(i) —S—CR¹²⁸=CR^{128′}—,

(j) —S—N═CH—,

(k) —CH═N—S—,

(l) —N═CR¹²⁸—O—,

(m) —O—CR4=N—,

(n) —N═CR¹²⁸-NH—,

(o) —N═CR¹²⁸-S—, and

(p) —S—CR¹²⁸═N—,

(q) —C(O)—NR¹²⁷-CR¹²⁹ (R^{129′})—,

(r) —R¹²⁷ N—CH═CH— provided R₁₂₂ is not —S(O)₂CH₃,

(s) —CH═CH—NR¹²⁷ _provided R¹²⁵ is not —S(O)₂CH₃,

when side b is a double bond, and sides a and c are single bonds; and

X¹⁷—Y¹—Z⁷— is selected from the group consisting of:

(a)=CH—O—CH═, and

(b)=CH—NR¹²⁷-CH═,

(c) ═N—S—CH═,

(d)=CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂ CH₃,

(b) S(O)₂ NH₂,

(c) S(O)₂ NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl,

wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹²⁹)(R¹³⁰)—OH,

(12) —C(R¹²⁹)(R³⁰)—O—C₁₋₄ alkyl, and

(13) —C₁₋₆ alkyl-CO₂-R¹²⁹,

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxyC₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C₁₋₃ alkyl-S—C₁₋₃ alkyl-Q⁵,

(4) —C₁₋₅ alkyl-O-Q⁵, or

(5) —C₁₋₅ alkyl-S-Q⁵,

wherein the substituent resides on the alkyl and the substituent is C₁₋₃ alkyl;

(h) -Q⁵;

R¹²⁸ and R^{128′} are each independently selected from the group consisting of:

(a) hydrogen,

(b) CF₃,

(c) CN,

(d) C₁₋₆ alkyl,

(e) -Q⁵,

(f) —O-Q⁵;

(g) —S-Q⁵, and

(h) optionally substituted:

(1) —C₁₋₅ alkyl-Q⁵,

(2) —O—C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O-Q⁵,

(7) —C₁₋₅ alkyl-S-Q⁵,

wherein the substituent resides on the alkyl and the substituent is C₁₋₃ alkyl, and

R¹²⁹, R¹²⁹, R¹³⁰, R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂H, CO₂—C₁₋₄ alkyl, tetrazolyl-5-yl, C(R¹³¹)(R¹³²)(OH), or C(R³¹)(R³²)(O—C₁₋₄ alkyl);

provided that when X-Y-Z is —S—CR¹²⁸=CR¹²⁸ then R¹²⁸ and R¹²⁸′ are other than CF₃.

Materials that can serve as a cyclooxygenase-2 selective inhibitor of the present invention include bicycliccarbonyl indole compounds that are described in U.S. Pat. No. 6,303,628. Such bicycliccarbonyl indole compounds have the formula shown below in formula XXV:

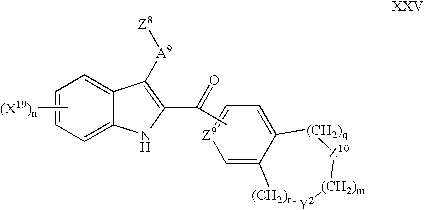

or the pharmaceutically acceptable salts thereof wherein

A⁹ is C₁₋₆ alkylene or —NR¹³³—;

Z⁸ is C(=L³)R¹³⁴ or SO₂ R¹³⁵;

Z⁹ is CH or N;

Z¹⁰ and Y² are independently selected from —CH₂—, O, S and —

N—R¹³³;

m is 1, 2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₁₄ alkyl(C₃₋₇ cycloalkoxy), —NR¹³⁶ R¹³⁷, C₄ alkylphenyl-O— or phenyl-O—, said phenyl being optionally substituted with one to five substituents independently selected from halogen, C₁₋₄ alkyl, hydroxy, C₁₋₄ alkoxy and nitro;

R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a cyclooxygenase-2 selective inhibitor of the present invention include benzimidazole compounds that are described in U.S. Pat. No. 6,310,079. Such benzimidazole compounds have the formula shown below in formula XXVI:

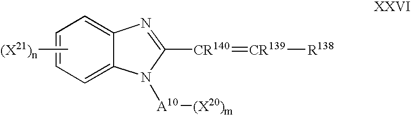

or a pharmaceutically acceptable salt thereof, wherein:

A¹⁰ is heteroaryl selected from a 5-membered monocyclic aromatic ring having one hetero atom selected from O, S and N and optionally containing one to three N atom(s) in addition to said hetero atom, or a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxy, C₁C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amonio, N-(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;

X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]c₁-C₄ alkyl, [N,N-di(C₁-C₄ alkyl)amino]C₁-C₄ alkyl, N-(C₁-C₄ alkanoyl)amino, N-(C₁-C₄ alkyl)-N-(C₁-C₄ alkanoyl) amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N-(C₁-C₄ alkyl) amino]carbonyl, [N,N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;

R¹³⁸ is selected from hydrogen,

straight or branched C₁-C₄ alkyl optionally substituted with one to three substituent(s) wherein said substituents are independently selected from halo hydroxy, C₁-C₄ alkoxy, amino, N-(C₁-C₄ alkyl)amino and N,N-di(C₁-C₄ alkyl)amino,

C₃-C₈ cycloalkyl optionally substituted with one to three substituent(s) wherein said substituents are indepently selected from halo, C₁-C₄ alkyl, hydroxy, C₁-C₄ alkoxy, amino, N-(C₁-C₄ alkyl)amino and N, N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N-(C₁-C₄ alkyl)amino and N,N-di(C₁-C₄ alkyl)amino,

phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-[C₁-C₄ alkyl)(C₁-C₄ alkanoyl)]amino, N-[(C₁-C₄ alkyl)sulfony]amino, N-[(halo-substituted C₁-C₄ alkyl)sulfonyl]amino, C₁-C₄ alkanoyl, carboxy, (C₁-C₄ alkoxy)carbonyl, carbomoyl, [N-(C₁-C₄ alky)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N-(C₁-C₄ alkyl)amino and N,N-di(C₁-C₄ alkyl)amino,

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Materials that can serve as a cyclooxygenase-2 selective inhibitor of the present invention include indole compounds that are described in U.S. Pat. No. 6,300,363. Such indole compounds have the formula shown below in formula XXVII:

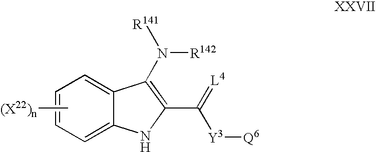

and the pharmaceutically acceptable salts thereof,

wherein:

L⁴ is oxygen or sulfur;

Y³ is a direct bond or C₁₋₄ alkylidene;

Q⁶ is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from: (c-1) halo, C₁₋₄ alkyl, halosubstituted C₁₋₄ alkyl, hydroxy, C₁₋₄ alkoxy, halosubstituted C₁₋₄ alkoxy, S(O)ₘ R¹⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³, CN, CO₂H, CO₂ (C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘ R⁴³, SO₂ NH₂, SO₂ N(C₁₋₄ alkyl)₂, amino, mono- or di-(C₁₋₄ alkyl)amino, NHSO₂ R¹⁴³, NHC(O)R¹⁴³ CN, CO₂H, CO₂ (C₁₋₄ alkyl), C₁₋₄ alkyl-OR⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂ CH₃, SO₂ NH₂, amino, C₁₋₄ alkylamino and NHSO₂ R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂ (C₁₋₄ alkyl), CONH₂, CONH(CO₁₄ alkyl) and CON(CO₁₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from: (c-1-1) halo, hydroxy, OR¹⁴³, S(O)ₘ R¹⁴³, nitro, amino, mono- or di-(C₁₋₄ alkyl)amino, NHSO₂ R¹⁴³, CO₂H, CO₂ (C₁₋₄ alkyl), CONH₂, CONH(C₁₋₄ alkyl), CON(C₁₋₄ alkyl)₂, OC(O)R¹⁴³, thienyl, naphthyl and groups of the following formulae:

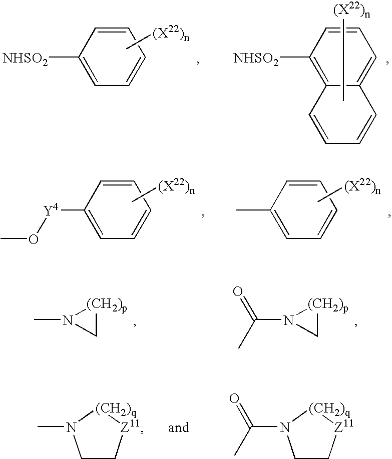

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘ R¹⁴³, amino, mono- or di-(C₁₋₄ alkyl)amino, CONH₂, CONH(C₁₋₄ alkyl) and CON(C₁₋₄ alkyl)₂, (c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘ R⁴³, SO₂ NH₂, SO₂ NH(C₁₋₄ alkyl), SO₂ N(C₁₋₄ alkyl)₂, amino, C₁₋₄ alkylamino, di-(C₁₋₄ alkyl)amino, CONH₂, CONH(CO₁₄ alkyl), CON(CO₁₄ alkyl)₂, OC(O)R¹⁴³, and phenyl optionally substituted with up to three substituents independently selected from halo, C₁₋₁₄ alkyl, hydroxy, OCH₃, CF₃, OCF₃, CN, nitro, amino, mono- or di-(C₁₋₄ alkyl)amino, CO₂H, CO₂ (C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘ R¹⁴³, amino, mono- or di-(C₁₋₄ alkyl)amino, CONH₂, CONH(C₁₋₄ alkyl), CON(C₁₋₄ alkyl)₂, CO₂H and CO₂ (C₁₋₄ alkyl), and —Y-phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘ R¹⁴³, amino, mono- or di-(C₁₋₄ alkyl)amino, CO₂H, CO₂ (C₁₋₄ alkyl), CONH₂, CONH(C₁₋₄ alkyl) and CON(C₁₋₄ alkyl)₂,

(c-6) a group of the following formula:

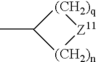

X²² is halo, C₁₋₄ alkyl, hydroxy, C₁₋₁₄ alkoxy, halosubstitutued C₁₋₄ alkoxy, S(O)ₘ R¹⁴³, amino, mono- or di-(C₁₋₄ alkyl)amino, NHSO₂ R¹⁴³, nitro, halosubstitutued C₁₋₄ alkyl, CN, CO₂H, CO₂ (C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂; R¹⁴³ is C₁₋₄ alkyl or halosubstituted C₁₋₄ alkyl;

m is 0, 1 or 2; n is 0, 1, 2 or 3; p is 1, 2, 3, 4 or 5; q is 2 or 3; Z¹¹ is oxygen, sulfur or NR¹⁴⁴; and

R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘ R¹⁴³, amino, mono- or di-(C₁₋₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Materials that can serve as a cyclooxygenase-2 selective inhibitor of the present invention include aryl phenylhydrazides that are described in U.S. Pat. No. 6,077,869. Such aryl phenylhydrazides have the formula shown below in formula XXVIII:

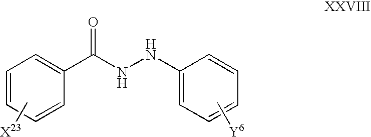

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a cyclooxygenase-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

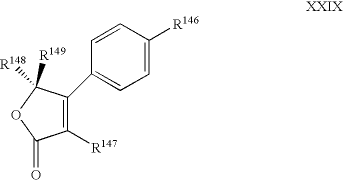

or a pharmaceutical salt thereof,

wherein:

R¹⁴⁶ is selected from the group consisting of SCH₃, —S(O)₂ CH₃ and —S(O)₂ NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted with 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a cyclooxygenase-2 selective inhibitor of the present invention include bisaryl compounds that are described in U.S. Pat. No. 5,994,379. Such bisaryl compounds have the formula shown below in formula XXX:

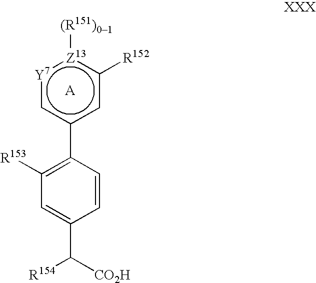

or a pharmaceutically acceptable salt, ester or tautomer thereof,

wherein:

Z¹³ is Cor N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 Ra group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a cyclooxygenase-2 selective inhibitor of the present invention include 1,5-diarylpyrazoles that are described in U.S. Pat. No. 6,028,202. Such 1,5-diarylpyrazoles have the formula shown below in formula X)XI:

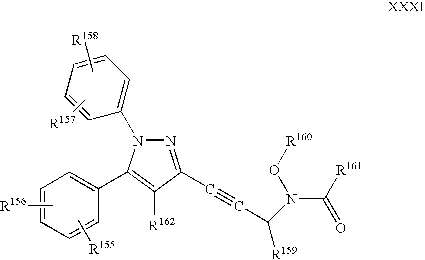

wherein:

R¹⁵⁵, R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³ R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen;

and pharmaceutically acceptable salts thereof.

Materials that can serve as a cyclooxygenase-2 selective inhibitor of the present invention include 2-substituted imidazoles that are described in U.S. Pat. No. 6,040,320. Such 2-substituted imidazoles have the formula shown below in formula XXXII:

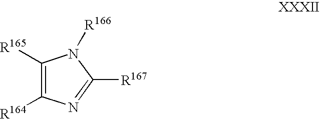

wherein:

R¹⁶⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms,

substituted heteroaryl;

wherein the substituents are independently selected from one or more members of the group consisting of C₁₋₅ alkyl and halogen, or substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅ alkyloxycarbonyl, arylC₁₋₅ alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅ alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ—(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl,

substituted sulfonamido,

wherein the sulfonyl substituent is selected from the group consisting of C₁₋₅ alkyl, phenyl, araC₁₋₅ alkyl, thienyl, furanyl, and naphthyl;

substituted vinyl,

wherein the substituents are independently selected from one or members of the group consisting of fluorine, bromine, chlorine and iodine,

substituted ethynyl,

wherein the substituents are independently selected from one or more members of the group consisting of fluorine, bromine chlorine and iodine,

substituted C₁₋₅ alkyl,

wherein the substituents are selected from the group consisting of one or more C₁₋₅ alkoxy, trihaloalkyl, phthalimido and amino,

substituted phenyl,

wherein the phenyl substituents are independently selected from one or more members of the group consisting of C₁₋₅ alkyl, halogen and C₁₋₅ alkoxy,

substituted phenoxy,

wherein the phenyl substituents are independently selected from one or more members of the group consisting of C₁₋₅ alkyl, halogen and C₁₋₅ alkoxy,

substituted C₁₋₅ alkoxy,

wherein the alkyl substituent is selected from the group consisting of phthalimido and amino,

substituted arylC₁₋₅ alkyl,

wherein the alkyl substituent is hydroxyl,

substituted arylC₁₋₅ alkyl,

wherein the phenyl substituents are independently selected from one or more members of the group consisting of C₁₋₅ alkyl, halogen and C₁₋₅ alkoxy,

substituted amido,

wherein the carbonyl substituent is selected from the group consisting of C₁₋₅ alkyl, phenyl, arylC₁₋₅ alkyl, thienyl, furanyl, and naphthyl, substituted phenylcarbonyl,

wherein the phenyl substituents are independently selected from one or members of the group consisting of C₁₋₅ alkyl, halogen and C₁₋₅ alkoxy,

substituted C₁₋₅ alkylthio,

wherein the alkyl substituent is selected from the group consisting of hydroxy and phthalimido,

substituted C₁₋₅ alkylsulfonyl,

wherein the alkyl substituent is selected from the group consisting of hydroxy and phthalimido,

substituted phenylsulfonyl,

wherein the phenyl substituents are independently selected from one or members of the group consisting of bromine, fluorine, chlorine, C₁₋₅ alkoxy and trifluoromethyl,

with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X²⁴ cannot be vinyl, ethynyl, C₁₋₅ alkylaminocarbonyl, phenylaminocarbonyl, arylC₁₋₅ alkylaminocarbonyl, C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen;

and pharmaceutically acceptable salts thereof.

Materials that can serve as a cyclooxygenase-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

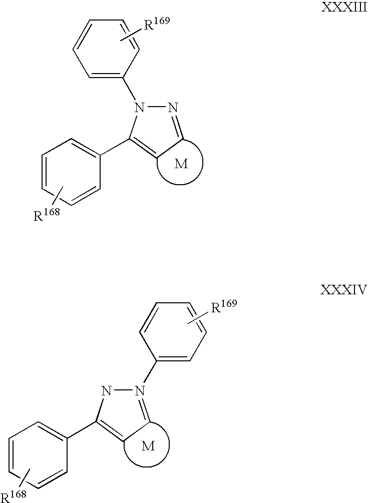

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂ (C₁-C₆)alkyl; and the fused moiety M is a group selected from the group consisting of an optionally substituted cyclohexyl and cycloheptyl group having the formulae:

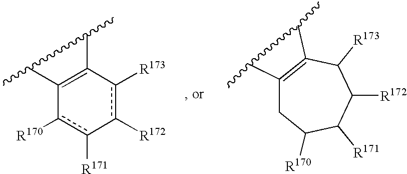

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ONH(CH₃)COCH₂—, —OCOCH.dbd. and —O—

R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴ R¹⁷⁵, —OCH₃, —OCH₂ CH₃, —OSO₂ NHCO₂ CH₃, ═CHCO₂ CH₂ CH₃, —CH₂ CO₂H, —CH₂ CO₂ CH₃, —CH₂ CO₂ CH₂ CH₃, —CH₂ CON(CH₃)₂, —CH₂ CO₂ NHCH₃, —CHCHCO₂ CH₂ CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

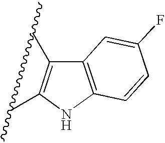

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂ CH₃; with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a cyclooxygenase-2 selective inhibitor of the present invention include esters derived from indolealkanois and novel amides derived from indolealkylamides that are described in U.S. Pat. No. 6,306,890. Such compounds have the general formula shown below in formula XXXV:

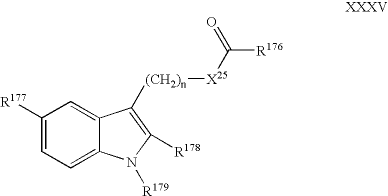

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C, to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₈ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo;

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C₁ to C₆ alkoxy, C₁ to C₆ branched alkoxy, C₄ to C₈ aryloxy, or halo-substituted versions thereof or R¹⁷⁷ is halo where halo is chloro, fluoro, bromo, or iodo;

R¹⁷⁸ is hydrogen, C₁ to C₆ alkyl or C, to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C, to C₆ branched alkyl.

Materials that can serve as a cyclooxygenase-2 selective inhibitor of the present invention include pyridazinone compounds that are described in U.S. Pat. No. 6,307,047. Such pyridazinone compounds have the formula shown below in formula XXXVI:

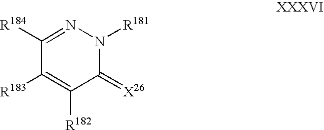

or a pharmaceutically acceptable salt, ester, or prodrug thereof,

wherein:

X²⁶ is selected from the group consisting of O, S, —NR¹⁸⁵, —NOR^{a}, and —NNR^{b} R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙ C(O)R¹⁸⁶, —(CH₂)ₙ CH(OH)R¹⁸⁶, —(CH₂)ₙ C(NORd)R¹⁸⁶, —(CH₂)ₙ CH(NORd)R¹⁸⁶, —(CH₂)ₙ CH(NRd Re)R¹⁸⁶, —R¹⁸⁷ R¹⁸⁸, —(CH₂)ₙ C□CR¹⁸⁸, —(CH₂)ₙ [CH(CX^{26′}₃)]ₘ (CH₂)ₚ R¹⁸⁸, —(CH₂)ₙ (CX²⁶^{₁}₂)ₘ (CH₂)ₚ R¹⁸⁸, and —(CH₂)ₙ (CHX²⁶^{₁}) (CH₂)ₘ R¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³ or R¹⁸⁴ must be Z¹⁴, and further provided that only one of R¹⁸², R¹⁸³, or R¹⁸⁴ is Z¹⁴;

Z¹⁴ is selected from the group consisting of:

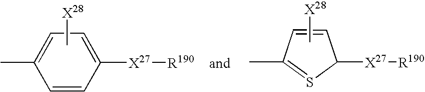

²⁷ is selected from the group consisting of S(O)₂, S(O)(NR¹⁹¹), S(O), Se(O)₂, P(O)(OR¹⁹²), and P(O)(NR¹⁹³ R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹ R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a cyclooxygenase-2 selective inhibitor of the present invention include benzosulphonamide derivatives that are described in U.S. Pat. No. 6,004,948. Such benzosulphonamide derivatives have the formula shown below in formula XXXVII:

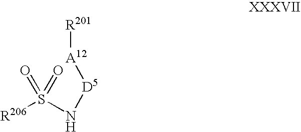

herein:

A¹² denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

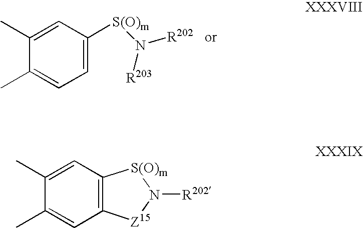

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R²⁰²′ denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂ R²⁰⁴, —OCO₂ R, —CN, —CONR²⁰⁴ OR²⁰⁵-CONR²⁰⁴ R²⁰⁵, —SR²⁰⁴ S(O)R²⁰⁴, —S(O)₂ R²⁰⁴, —NR²⁰⁴ R²⁰⁵, —NHC(O)R²⁰⁴, —NHS(O)₂ R²⁰⁴; Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂ NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄-alkyl group which may optionally be mono- or polysubstituted by halogen or alkoxy, or R²⁰⁶ denotes CF₃; and

m denotes an integer from 0 to 2;

with the proviso that A¹² does not represent 0 if R²⁰⁶ denotes CF₃;

and the pharmaceutically acceptable salts thereof.

Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

Another component of the present invention is a colds and cough active ingredient. It is preferred that the colds and cough active ingredient is different than the cyclooxygenase-2 selective inhibitor. In general, colds and cough medications can be used to relieve the cough and other symptoms due to colds, influenza, or hay fever. Commonly, two or more ingredients that have activity against the same or different symptoms of colds or coughs can be used together in a combination. As these terms are used herein, “colds and cough active ingredient” is meant to include any element, compound or material, alone or in combination, that has been used for, or has been shown to be useful for, the prevention, treatment or amelioration of at least one symptom commonly associated with colds or cough. Examples of general categories of colds and cough active ingredients include antihistamines, decongestants, antitussives, expectorants, analgesics, anticholinergics and antiviral agents. It should be understood that when any colds and cough active ingredient is referred to herein, all pharmaceutically acceptable salts and prodrugs of the material are also included unless specified otherwise.

Antihistamines are used to relieve or prevent the symptoms of hay fever and other types of allergy. They also help relieve some symptoms of the common cold, such as sneezing and runny nose. They work by preventing the effects of histamine, which is produced by the body. Some examples of antihistamines are: bromodiphenhydramine, brompheniramine, carbinoxamine, chlorpheniramine, dexchlorpheniramine, diphenhydramine, doxylamine, phenindamine, pheniramine, phenyltoloxamine, pyrilamine, promethazine, triprolidine, loratadine, and cetirzine.

Decongestants, such as ephedrine, phenylephrine, phenylpropanolamine and pseudoephedrine, produce a narrowing of blood vessels. This leads to clearing of nasal congestion.

Antitussives help relieve coughing. Examples of antitussives include those which are narcotics, such as codeine, dihydrocodeine, hydrocodone and hydromorphone, or a non-narcotic, such as carbetapentane, caramiphen, or dextromethorphan. It is believed that antitussives act directly on the cough center in the brain.

Expectorants, such as guaifenesin, are believed to work by loosening the mucus or phlegm in the lungs. Examples of other ingredients that are added as expectorants include ammonium chloride, calcium iodide, iodinated glycerol, ipecac, potassium guaiacolsulfonate, potassium iodide, and sodium citrate.

Analgesics, such as acetaminophen, aspirin, and other salicylates, such as salicylamide and sodium salicylate, are used to help relieve the aches and pain that may occur with the common cold.

Anticholinergics such as homatropine, may help produce a drying effect in the nose and chest.

Antiviral agents specifically or generally modulate the biological activity of viruses such as picornavirus, influenza virus, herpesviruses, herpes simples, herpes zoster, enteroviruses, varicella and rhinovirus, which are associated with the common cold. Examples of antiviral agents include neuramimidase inhibitors such as zanamivir and oseltamivir; agents for herpesviruses such as famciclovir, valaciclovir, valganciclovir, aciclovir and ganciclovir; interferons; interferon-inducers; and newer antiviral agents such as dipyridamole; ICI 130,685; impulsin; and pleconaril (VP-63843; 3-[3,5-dimethyl-4[[3-(3-methyl-5-isoxazolyl)propyl]oly]phenyl]-5-(trifluoromethyl)-1,2,4-oxad iazole; available under the tradename PICOVIR® from ViroPharma and Sanofi-Synthelabo).

Other materials can be used along with the subject combination of a Cox-2 selective inhibitor and at least one colds and cough active ingredient. For example, ingredients such as caffeine, potassium citrate, ascorbic acid and citric acid can be added to the combinations, as can such materials as fillers, dyes, binders, adsorbents, surfactants, and the like.

One embodiment of the present invention is a composition that includes a cycloxygenase-2 selective inhibitor and one or more colds and cough active ingredient. Any one of, or any combination of, the Cox-2 selective inhibitors that are described above can be used in the composition. Likewise, the colds and cough active ingredient can be selected from an antihistamine, antitussive, analgesic, expectorant, decongestant, anticholinergic, antiviral agent, or a mixture of two or more thereof.

In an embodiment, the colds and cough active ingredient comprises an antihistamine. It is preferred that the antihistamine is selected from the group consisting of azatadine, bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, promethazine, pyrilamine, triprolidine, and mixtures thereof.

In another embodiment, the colds and cough active ingredient comprises an antitussive. In preferred embodiments, the antitussive is selected from the group consisting of codeine, dihydrocodeine, hydrocodone, hydrocodone bitartrate, hydromorphone, carbetapentane, caraminphen, dextromethorphan, chlorphedianol, noscarpine, and mixtures thereof.

In another embodiment, the colds and cough active ingredient comprises an analgesic. It preferred embodiments, the analgesic is selected from the group consisting of acetaminophen, aspirin, salicylamide, sodium salicylate, indomethacin, ibuprofen, naproxen, flubiprofen, carprofen, tiaprofenic acid, cicloprofen, detoprofen, ketorolac, etodolac, and mixtures thereof.

In another embodiment, the colds and cough active ingredient comprises an expectorant. In preferred embodiments, the expectorant comprises guaifenesin, glyceryl guaiacolate, terpin hydrate, ammonium chloride, N-acetylesteine, bromhexine, ambroxol, domiodol, 3-iodo-1,2-propanediol, and mixtures thereof.

In another embodiment, the colds and cough active ingredient comprises a decongestant. In preferred embodiments, the decongestant is selected from the group consisting of ephedrine, ephinephrine, levodesoxyephedrine, oxymetazoline, naphazoline, phenylephrine, phenylpropanolamine, propylhexedrine, pseudoephedrine, xylometazoline, and mixtures thereof.

In another embodiment, the colds and cough active ingredient comprises an anticholinergic. In preferred embodiments, the anticholinergic comprises homatropine.

In another embodiment, the colds and cough active ingredient comprises an antiviral agent. In preferred embodiments, the antiviral agent comprises a neuramimidase inhibitor, an agent for herpesviruses, an interferon, or an interferon-inducer. In more preferred embodiments, the antiviral agent comprises dipyridamole, ICI 130,685, impulsin, pleconaril, zanamivir, oseltamivir, famciclovir, valaciclovir, valganciclovir, aciclovir (acyclovir), ganciclovir, idoxuridine, vidarabine, trifluridine, penciclovir, valacyclovir, foscarnet, ribavarin, amantadine, rimantadine, cidofovir, or two or more of these compounds.

Another embodiment of the present invention is a composition that includes a cycloxygenase-2 selective inhibitor and two or more different types of colds and cough active ingredients. The Cox-2 selective inhibitor can be any one of, or any combination of, the Cox-2 selective inhibitors that are described above. The two or more different types of colds and cough active ingredients can be selected from any combination of two or more of an antihistamine, antitussive, analgesic, expectorant, decongestant, anticholinergic, or an antiviral agent. Preferred embodiments of the present invention include a cyclooxygenase-2 selective inhibitor in combination with any of the combinations of two or more colds and cough active ingredients that are shown in Table 3.

Table 3: Combinations of two or more colds and cough active ingredients and trade names of commercial compositions that include the combination.

| | | COLDS AND COUGH ACTIVE INGREDIENT |
|---|---|---|
| NUMBER | TRADE-NAME | COMBINATIONS |
| 1 | | An antihistamine and an antitussive. |
| 2 | Ambenyl Cough; | bromodiphenhydramine and codeine. |
| | Ambophen; | |
| | Amgenal Cough; | |
| | Bromanyl; | |
| | Bromotuss with | |
| | Codeine; | |
| 3 | | chlorpheniramine and codeine. |
| 4 | Effective Strength | chlorpheniramine and dextromethorphan. |
| | Cough Formula; | |
| | Primatuss Cough | |
| | Mixture; | |
| | Scot-Tussin DM; | |
| | Tricodene Sugar Free; | |
| 5 | S-T-Forte 2; | chlorpheniramine and hydrocodone. |
| | Tussionex | |
| | Pennkinetic; | |
| 6 | | phenyltoloxamine and hydrocodone. |
| 7 | Pentazine VC w/ | promethazine and codeine. |
| | Codeine; | |
| | Phenergan with | |
| | Codeine; | |
| | Pherazine w/Codeine; | |
| 8 | Phenameth DM; | promethazine and dextromethorphan. |
| | Phenergan with | |
| | Dextromethorphan; | |
| | Pherazine DM; | |
| | Promethazine DM; | |
| | Prometh w/ | |
| | Dextromethorphan; | |
| 9 | Tricodene; | pyrilamine and codeine. |
| 10 | | An antihistamine, an antitussive, and an |
| | | analgesic. |
| 11 | | doxylamine, codeine and acetaminophen. |
| 12 | | An antihistamine, an antitussive, and an |
| | | expectorant. |
| 13 | | bromodiphenhydramine, diphenhydramine, |
| | | codeine, ammonium chloride and potassium |
| | | guaiacolsulfonate. |
| 14 | | diphenhydramine, codeine and ammonium |
| | | chloride. |
| 15 | | diphenhydramine, dextromethorphan and |
| | | ammonium chloride. |
| 16 | | pheniramine, codeine and guaifenesin. |
| 17 | Citra-Forte; | pheniramine, pyrilamine, hydrocodone, |
| | | potassium citrate and ascorbic acid. |
| 18 | Citra-Forte; | chlorpheniramine, pheniramine, pyrilamine, |
| | | phenylephrine, hydrocodone salicylamide, |
| | | caffeine and ascorbic acid. |
| 19 | | promethazine, codeine and potassium |
| | | guaiacolsulfonate. |
| 20 | | An antihistamine, a decongestant and an |
| | | antitussive. |
| 21 | | brompheniramine, phenylephrine, |
| | | phenylpropanolamine and codeine. |
| 22 | | brompheniramine, phenylephrine, |
| | | phenylpropanolamine and dextromethorphan. |
| 23 | Bromonate DC Cough; | brompheniramine, phenylpropanolamine and |
| | Bromphen DC with | codeine. |
| | Codeine Cough; | |
| | Dimetane-DC Cough; | |
| | Myphetane DC Cough; | |
| | Poly-Histine-CS; | |
| 24 | Dimetapp DM; | brompheniramine, phenylpropanolamine and |
| | Dimetapp DM Cold & | dextromethorphan. |
| | Cough; | |
| | Dimetapp Maximum | |
| | Strength Cold & | |
| | Cough Liqui-Gels; | |
| | Histinex DM; | |
| | lohist DM; | |
| | Liqui-Histine DM; | |
| | Poly-Histine-DM; | |
| | Siltapp w/ | |
| | Dextromethorphan | |
| | Cough & Cold; | |
| 25 | Bromarest DX Cough; | brompheniramine, pseudoephedrine and |
| | Bromatene DX Cough; | dextromethorphan. |
| | Bromfed DM; | |
| | Bromphen DX Cough; | |
| | Brotane DX Cough; | |
| | Dimetane-DX Cough; | |
| | Myphetane DX Cough; | |
| 26 | Carbinoxamine | carbinoxamine, pseudoephidrine and |
| | Compound; | dextromethorphan. |
| | Carbinoxamine | |
| | Compound Drops; | |
| | Carbodec DM; | |
| | Carbodec DM Drops; | |
| | Cardec DM; | |
| | Cardec DM Drops; | |
| | Cardec DM Pediatric; | |
| | Pseudo-Car DM; | |
| | Rondamine-DM | |
| | Drops; | |
| | Rondec-DM; | |
| | Rondec-DM Drops; | |
| | Sildec-DM; | |
| | Sildec-DM Oral Drops; | |
| | Tussafed; | |
| | Tussafed Drops; | |
| 27 | Rentamine Pediatric; | chlorpheniramine, ephedrine, phenylephrine |
| | Rynatuss; | and carbetapentane. |
| | Rynatuss Pediatric; | |
| | Tri-Tannate Plus | |
| | Pediatric; | |
| 28 | Atuss DM; | chlorpheniramine, phenylephrine and |
| | Cerose DM; | dextromethorphan. |
| | Dondril; | |
| 29 | Anaplex HD; | chlorpheniramine, phenylephrine and |
| | Atuss HD; | hydrocodone. |
| | Chlorgest-HD; | |
| | ED-TLC; | |
| | ED Tuss HC; | |
| | Edagen-HD; | |
| | Endal-HD; | |
| | Endal-HD Plus; | |
| | Histinex HC; | |
| | Histussin HC; | |
| | lodal HD; | |
| | lotussin HC; | |
| | Med-Hist HC; | |
| | Nasatuss; | |
| | Para-Hist HD; | |
| | Unituss HC; | |
| | Vanex-HD; | |
| 30 | T-Koff; | chlorpheniramine, phenylephrine, |
| | | phenylpropanolamine and codeine. |
| 31 | Cophene-S; | chlorpheniramine, phenylephrine, |
| | Vanex Grape; | phenylpropanolamine and dihydrocodeine. |
| 32 | | chlorpheniramine, phenylpropanolamine and |
| | | caramiphen. |
| 33 | Cheracol Plus; | chlorpheniramine, phenylpropanolamine and |
| | Kophane Cough and | dextromethorphan. |
| | Cold Formula; | |
| | Myminicol; | |
| | Snaplets-Multi; | |
| | Threamine DM; | |
| | Triaminicol Multi- | |
| | Symptom Cold and | |
| | Cough Medicine; | |
| | Triaminic Triaminicol; | |
| | Tricodene Forte; | |
| | Tricodene NN; | |
| | Triminol Cough; | |
| 34 | Codehist DH; | chlorpheniramine, pseudoephedrine and |
| | Decohistine DH; | codeine. |
| | Dihistine DH; | |
| | Medahist DH; | |
| | Novahistine DH Liquid; | |
| | Phenhist DH w/ | |
| | Codeine; | |
| | Ryna-C Liquid; | |
| 35 | PediaCare Cough- | chlorpheniramine, pseudoephedrine and |
| | Cold; | dextromethorphan. |
| | PediaCare Night Rest | |
| | Cough-Cold Liquid; | |
| | Rescon-DM; | |
| | Rhinosyn-DM; | |
| | Triaminic Night Time; | |
| | Tussar DM; | |
| | Vicks Children's | |
| | NyQuil Cold/Cough | |
| | Relief; | |
| | Vicks Pediatric 44 M | |
| | Multi-Symptom Cough | |
| | & Cold; | |
| 36 | Histinex PV; | chlorpheniramine, pseudoephedrine and |
| | Promist HD Liquid; | hydrocodone. |
| | P-V-Tussin; | |
| 37 | | diphenylpyraline, phenylephrine and |
| | | dextromethorphan. |
| 38 | | doxylamine, etafedrine and hydrocodone. |
| 39 | | pheniramine, phenylephrine and |
| | | dextromethorphan. |
| 40 | Rolatuss w/ | pheniramine, pyrilamine, phenylephrine, |
| | Hydrocodone; | phenylpropanolamine and hydrocodone. |
| | Ru-Tuss with | |
| | Hydrocodone Liquid; | |
| | Statuss Green; | |
| 41 | | pyrilamine, phenylpropanolamine and codeine. |
| 42 | | pheniramine, pyrilamine, phenylpropanolamine |
| | | and dextromethorphan. |
| 43 | | pheniramine, pyrilamine, phenylpropanolamine |
| | | and hydrocodone. |
| 44 | Phenameth VC with | promethazine, phenylephrine and codeine. |
| | Codeine; | |
| | Phenergan VC with | |
| | Codeine; | |
| | Pherazine VC with | |
| | Codeine; | |
| | Promethazine VC w/ | |
| | Codeine; | |
| | Promethist w/ | |
| | Codeine; | |
| | Prometh VC with | |
| | Codeine; | |
| 45 | | promethazine, pseudoephedrine and |
| | | dextromethorphan. |
| 46 | Codimal PH; | pyrilamine, phenylephrine and codeine. |
| 47 | Codimal DM; | pyrilamine, phenylephrine and |
| | | dextromethorphan. |
| 48 | Codimal DH; | pyrilamine, phenylephrine and hydrocodone. |
| 49 | Actagen-C-Cough; | triprolidine, pseudoephedrine and codeine. |
| | Actifed with Codeine | |
| | Cough; | |
| | Allerfrin with Codeine; | |
| | Aprodine with | |
| | Codeine; | |
| | Triacin C Cough; | |
| | Triafed w/ Codeine; | |
| | Trifed-C Cough; | |
| 50 | | triprolidine, pseudoephedrine and |
| | | dextromethorphan. |
| 51 | | An antihistamine, a decongestant, an |
| | | antitussive and an analgesic. |
| 52 | Omnicol; | chlorpheniramine, phenindamine, |
| | | phenylephrine, dextromethorphan, |
| | | acetaminophen, salicylamide, caffeine and |
| | | ascorbic acid. |
| 53 | | chlorpheniramine, pheniramine, pyrilamine, |
| | | phenylephrine, hydrocodone, salicylamide, |
| | | caffeine and ascorbic acid. |
| 54 | Improved Sino-Tuss; | chlorpheniramine, phenylephrine, |
| | | dextromethorphan, acetaminophen and |
| | | salicylamide. |
| 55 | Hycomine Compound; | chlorpheniramine, phenylephrine, |
| | | hydrocodone, acetaminophen and caffeine. |
| 56 | Alka-Seltzer Plus Flu | chlorpheniramine, phenylpropanolamine, |
| | & Body Aches; | dextromethorphan and acetaminophen. |
| | Comtrex Maximum | |
| | Strength Multi- | |
| | Symptom Liqui-Gels; | |
| | Comtrex Multi- | |
| | Symptom Cold | |
| | Reliever; | |
| | Contac Severe Cold & | |
| | Flu Caplets; | |
| 57 | Alka-Seltzer Plus Cold | chlorpheniramine, phenylpropanolamine, |
| | and Cough; | dextromethorphan and aspirin. |
| 58 | | chlorpheniramine, pseudoephedrine, codeine |
| | | and acetaminophen. |
| 59 | Alka-Seltzer Plus Cold | chlorpheniramine, pseudoephedrine, |
| | and Cough Medicine | dextromethorphan and acetaminophen. |
| | Liqui-Gels; | |
| | Children's Tylenol | |
| | Cold Plus Cough Multi | |
| | Symptom; | |
| | Comtrex Nighttime; | |
| | Comtrex Nighttime | |
| | Maximum Strength | |
| | Cold, Cough and Flu | |
| | Relief; | |
| | Comtrex Nighttime | |
| | Maximum Strength | |
| | Cold and Flu Relief; | |
| | Kolephrin/DM Cough | |
| | and Cold Medication; | |
| | Mapap Cold Formula; | |
| | TheraFlu Flu, Cold & | |
| | Cough Medicine; | |
| | TheraFlu Nighttime | |
| | Maximum Strength | |
| | Flu, Cold & Cough; | |
| | Tylenol Cold | |
| | Medication; | |
| | Tylenol Cold | |
| | Medication Caplets; | |
| | Tylenol Cold Multi- | |
| | Symptom; | |
| | Vicks 44 M Cough, | |
| | Cold and Flu Relief; | |
| | Vicks 44 M Cough, | |
| | Cold and Flu Relief | |
| | LiquidCaps; | |
| 60 | Alka-Seltzer Plus | doxylamine, phenylpropanolamine, |
| | Night-Time Cold; | dextromethorphan and aspirin. |
| | Co-Apap; | |
| 61 | Alka-Seltzer Plus | doxylamine, pseudoephedrine, |
| | Night-Time Cold Liqui- | dextromethorphan and acetaminophen. |
| | Gels; | |
| | All-Night Cold | |
| | Formula; | |
| | Genite; | |
| | Nytcold Medicine; | |
| | Robitussin Night-Time | |
| | Cold Formula; | |
| | Vicks NyQuil Hot | |
| | Therapy; | |
| | Vicks NyQuil Multi- | |
| | Symptom Cold/Flu | |
| | LiquiCaps; | |
| | Vicks NyQuil Multi- | |
| | Symptom Cold/Flu | |
| | Relief; | |
| 62 | Robitussin Night | pyrilamine, pseudoephedrine, |
| | Relief; | dextromethorphan and acetaminophen. |
| 63 | | An antihistamine, a decongestant, an |
| | | antitussive and an expectorant. |
| 64 | | brompheniramine, phenylephrine, |
| | | phenylpropanolamine, codeine and |
| | | guaifenesin. |
| 65 | | comprises brompheniramine, phenylephrine, |
| | | phenylpropanolamine, hydrocodine and |
| | | guaifenesin. |
| 66 | Quelidrine Cough; | chlorpheniramine, ephedrine, phenylephrine, |
| | | dextromethorphan, ammonium chloride and |
| | | ipecac. |
| 67 | Tusquelin; | chlorpheniramine, phenylephrine, |
| | | phenylpropanolamine, dextromethorphan, |
| | | potassium guaiacolsulfonate and ipecac. |
| 68 | Rolatuss Expectorant; | chlorpheniramine, phenylephrine, codeine and |
| | | ammonium chloride. |
| 69 | Pediacof Cough; | chlorpheniramine, phenylephrine, codeine and |
| | Pedituss Cough; | potassium iodide. |
| 70 | Donatussin; | chlorpheniramine, phenylephrine, |
| | | dextromethorphan and guaifenesin. |
| 71 | Father John' Medicine | chlorpheniramine, phenylephrine, |
| | Plus; | dextromethorphan, guaifenesin and ammonium |
| | | chloride. |
| 72 | Cophene-XP; | chlorpheniramine, phenylephrine, |
| | | phenylpropanolamine, carbetapentane and |
| | | potassium guaiacolsulfonate. |
| 73 | | chlorpheniramine, phenyltoloxamine, |
| | | ephedrine, codeine and guaiacol carbonate. |
| 74 | | chlorpheniramine, pseudoephedrine, |
| | | dextromethorphan and guaifenesin. |
| 75 | Prominicol Cough; | pheniramine, pyrilamine, phenylpropanolamine, |
| | | dextromethorphan and ammonium chloride. |
| 76 | Triaminic Expectorant | pheniramine, pyrilamine, phenylpropanolamine, |
| | DH | hydrocodone and guaifenesin. |
| 77 | S-T-Forte; | pheniramine, phenylephrine, |
| | | phenylpropanolamine, hydrocodone and |
| | | guaifenesin. |
| 78 | | promethazine, phenylephrine, codeine and |
| | | potassium guaiacolsulfonate. |
| 79 | | pyrilamine, phenylephrine, hydrocodone and |
| | | ammonium chloride. |
| 80 | Phanatussin; | pyrilamine, phenylpropanolamine, |
| | | dextromethorphan and guaifenesin. |
| 81 | | triprolidine, pseudoephedrine, codeine and |
| | | guaifenesin. |
| 82 | | An antihistamine, a decongestant, an |
| | | antitussive, an expectorant and an |
| | | analgesic. |
| 83 | Tussirex; | pheniramine, phenylephrine, codeine, sodium |
| | | citrate, sodium salicylate and caffeine. |
| 84 | | An antihistamine, a decongestant and an |
| | | expectorant. |
| 85 | | brompheniramine, phenylephrine, |
| | | phenylpropanolamine and guaifenesin. |
| 86 | Bronkotuss | chlorpheniramine, ephedrine and guaifenesin. |
| | Expectorant; | |
| 87 | Donatussin Drops; | chlorpheniramine, phenylephrine and |
| | | guaifenesin. |
| 88 | | chlorpheniramine, phenylpropanolamine and |
| | | guaifenesin. |
| 89 | Lanatuss Expectorant; | chlorpheniramine, phenylpropanolamine, |
| | | guaifenesin, sodium citrate and citric acid. |
| 90 | | chlorpheniramine, pseudoephedrine and |
| | | guaifenesin. |
| 91 | Polaramine | dexchlorpheniramine, pseudoephedrine and |
| | Expectorant; | guaifenesin. |
| 92 | | promethazine, phenylephrine and potassium |
| | | guaiacolsulfonate. |
| 93 | | An antihistamine, a decongestant, an |
| | | expectorant and an analgesic. |
| 94 | Gelpirin-CCF; | chlorpheniramine, phenylpropanolamine, |
| | | guaifenesin and acetaminophen. |
| 95 | | An antihistamine and an expectorant. |
| 96 | Drixoral Cough & Sore | promethazine and potassium |
| | Throat Liquid Caps; | guaiacolsulfonate. |
| | Tylenol Multi-Symptom | |
| | Cough; | |
| 97 | | An antitussive and an analgesic. |
| 98 | | dextromethorphan and acetaminophen. |
| 99 | | An antitussive and an antichlolinergic. |
| 100 | Codan; | hydrocodone and homatropine. |
| | Hycodan; | |
| | Hydromet; | |
| | Hydropane; | |
| | Tussigon; | |
| 101 | | An antitussive and an expectorant. |
| 102 | Cheracol; | codeine, ammonium chloride and guaifenesin. |
| 103 | Calcidrine; | codeine and calcium iodide. |
| 104 | Brontex; | codeine and guaifenesin. |
| | Glydeine Cough; | |
| | Guaituss A.C.; | |
| | Mytussin AC; | |
| | Robafen AC Cough; | |
| | Robitussin A-C; | |
| | Tolu-Sed Cough; | |
| | Tussi-Organidin NR | |
| | Liquid; | |
| | Tussi-Organidin-S NR | |
| | Liquid; | |
| 105 | lophen-C Liquid; | codeine and iodated glycerol. |
| 106 | Anti-Tuss DM | dextromethorphan and guaifenesin. |
| | Expectorant; | |
| | Benylin Expectorant; | |
| | Cheracol D Cough; | |
| | Children's Formula | |
| | Cough; | |
| | Diabetic Tussin DM; | |
| | Extra Action Cough; | |
| | Fenesin DM; | |
| | Genatuss DM; | |
| | Glycotuss-dM; | |
| | Guaimid D.M. Liquid; | |
| | Guaitussin w/ | |
| | Dextromethorphan; | |
| | Halotussin-DM; | |
| | Humibid DM; | |
| | Humibid DM Pediatric; | |
| | lobid DM; | |
| | Kolephrin GG/DM; | |
| | Muco-Fen DM; | |
| | Mytussin DM; | |
| | Naldecon Senior DX; | |
| | Phanatuss; | |
| | Respa-DM; | |
| | Rhinosyn-DMX | |
| | Expectorant; | |
| | Robafen DM; | |
| | Robitussin-DM; | |
| | Safe Tussin 30; | |
| | Scot-Tussin Senior | |
| | Clear; | |
| | Silexin Cough; | |
| | Siltussin-DM; | |
| | Supressin DM; | |
| | Supressin DM | |
| | Caplets; | |
| | Syracol CF; | |
| | Tolu-Sed DM | |
| | Touro DM; | |
| | Tuss-DM; | |
| | Tussi-Organidin DM | |
| | NR Liquid; | |
| | Tussi-Organidin DM-S | |
| | NR Liquid; | |
| | Uni-tussin DM; | |
| | Unproco; | |
| | Vicks 44E Cough & | |
| | Chest Congestion; | |
| | Vicks Pediatric 44E; | |
| 107 | lophen DM; | dextromethorphan and iodated glycerol. |
| | Tusso-DM; | |
| 108 | Atuss EX; | hydrocodone and guaifenesin. |
| | Codiclear DH; | |
| | Co-Tuss V; | |
| | Hycotuss Expectorant; | |
| | Kwelcof Liquid; | |
| | Pneumotussin HC; | |
| | Vicodin Tuss; | |
| 109 | Entuss Expectorant; | hydrocodone and potassium guaiacolsulfonate. |
| | Marcof Expectorant; | |
| 110 | Dilaudid Cough; | hydromorphone and guaifenesin. |
| 111 | | A decongestant and an antitussive. |
| 112 | | phenylephrine and codeine. |
| 113 | Nalex DH; | phenylephrine and hydrocodone. |
| 114 | Ordrine AT; | phenylpropanolamine and caramiphen. |
| | Rescaps-D S.R.; | |
| | Tuss-Ade; | |
| | Tuss-Allergine | |
| | Modified T.D.; | |
| | Tussogest; | |
| 115 | Snaplets-DM; | phenylpropanolamine and dextromethorphan. |
| | Triaminic-DM Cough | |
| | Relief; | |
| | Tricodene Pediatric; | |
| 116 | Codamine; | phenylpropanolamine and hydrocodone. |
| | Codamine Pediatric | |
| | Hycomine; | |
| | Hycomine Pediatric; | |
| | Hydromine; | |
| | Hydromine Pediatric; | |
| | Hydrophen; | |
| 117 | Nucofed; | pseudoephedrine and codeine. |
| 118 | Drixoral Cough & | pseudoephedrine and dextromethorphan. |
| | Congestion Liquid | |
| | Caps; | |
| | Effective Strength | |
| | Cough Formula with | |
| | Decongestant; | |
| | Robitussin Maximum | |
| | Strength Cold and | |
| | Cough; | |
| | Robitussin Pediatric | |
| | Cold & Cough; | |
| | Triaminic AM Non- | |
| | Drowsy Cough and | |
| | Decongestant; | |
| | Tuss-DA; | |
| | Vicks 44 Cough and | |
| | Cold Relief Non- | |
| | Drowsy LiquiCaps; | |
| | Vicks 44D Cough and | |
| | Head Congestion; | |
| | Vicks Pediatric 44D | |
| | Cough & Head | |
| | Decongestion; | |
| 119 | De-Tuss; | pseudoephedrine and hydrocodone. |
| | Detussin Liquid; | |
| | Tyrodone; | |
| 120 | | A decongestant, an antitussive and an |
| | | analgesic. |
| 121 | Saleto-CF; | phenylpropanolamine, dextromethorphan and |
| | | acetaminophen. |
| 122 | Alka-Seltzer Plus Flu | pseudoephedrine, dextromethorphan and |
| | & Body Aches | acetaminophen. |
| | Medicine Liqui-Gels; | |
| | Co-Complex DM | |
| | Caplets; | |
| | Comtrex Daytime | |
| | Caplets; | |
| | Contrex Daytime | |
| | Maximum Strength | |
| | Cold, Cough, and Flu | |
| | Relief; | |
| | Comtrex Daytime | |
| | Maximum Strength | |
| | Cold and Flu Relief; | |
| | Comtrex Multi- | |
| | Symptom Maximum | |
| | Strength Non-Drowsy | |
| | Caplets; | |
| | Contac Cold/Flu Day | |
| | Caplets; | |
| | Contac Severe Cold & | |
| | Flu Non-Drowsy | |
| | Caplets; | |
| | Ornex Severe Cold No | |
| | Drowsiness Caplets; | |
| | Sudafed Severe Cold | |
| | Formula; | |
| | Sudafed Severe Cold | |
| | Formula Caplets; | |
| | TheraFlu Maximum | |
| | Strength Non-Drowsy | |
| | Formula Flu, Cold & | |
| | Cough Medicine; | |
| | TheraFlu Maximum | |
| | Strength Non-Drowsy | |
| | Formula Flu, Cold & | |
| | Cough Medicine | |
| | Caplets; | |
| | Triaminic Sore Throat | |
| | Formula; | |
| | Tylenol Cold and Flu | |
| | Non Crowsiness | |
| | Powder; | |
| | Tylenol Cold | |
| | Medication, Non- | |
| | Drowsy Caplets; | |
| | Tylenol Cold | |
| | Medication, Non- | |
| | Drowsy Caplets; | |
| | Tylenol Maximum | |
| | Strength Flu Gelcaps; | |
| | Tylenol Multi-Symptom | |
| | Cough with | |
| | Decongestant; | |
| 123 | | A decongestant, an antitussive and an |
| | | expectorant. |
| 124 | | ephedrine, carbetapentane and guaifenesin. |
| 125 | Dexafed Cough; | phenylephrine, dextromethorphan and |
| | Supressin DM Plus; | guaifenesin. |
| | Tussex Cough; | |
| 126 | Cophene-X; | phenylephrine, phenylpropanolamine, |
| | | carbetapentane and potassium |
| | | guaiacolsulfonate. |
| 127 | Donatussin DC; | phenylephrine, hydrocodone and guaifenesin. |
| 128 | Codegest Expectorant; | phenylpropanolamine, codeine and |
| | Conex with Codeine | guaifenesin. |
| | Liquid; | |
| | C-Tussin Expectorant; | |
| | Endal Expectorant; | |
| | Naldecon-CX Adult | |
| | Liquid; | |
| | Statuss Expectorant; | |
| | Triaminic Expectorant | |
| | with Codeine; | |
| 129 | Anatuss; | phenylpropanolamine, dextromethorphan and |
| | GuaiCough CF; | guaifenesin. |
| | Guaituss CF; | |
| | Ipsatol Cough Formula | |
| | for Children and | |
| | Adults; | |
| | Kiddy Koff; | |
| | Naldecon-DX Adult | |
| | Liquid; | |
| | Naldecon-DX | |
| | Children's Syrup; | |
| | Naldecon-DX Pediatric | |
| | Drops; | |
| | Robafen CF; | |
| | Robitussin-CF; | |
| | Siltussin-CF; | |
| 130 | Anatuss; | phenylpropanolamine, dextromethorphan, |
| | | guaifenesin and acetaminophen. |
| 131 | Deproist Exectorant | pseudoephedrine, codeine and guaifenesin. |
| | with Codeine; | |
| | Dihistine Expectorant; | |
| | Guaituss DAC; | |
| | Mytussin DAC; | |
| | Novagest Expectorant | |
| | w/Codeine; | |
| | Novahistine | |
| | Expectorant; | |
| | Nucochem | |
| | Expectorant; | |
| | Nucochem Pediatric | |
| | Expectorant; | |
| | Nucofed Expectorant; | |
| | Nucofed Pediatric | |
| | Expectorant; | |
| | Nucotuss Expectorant; | |
| | Nucotuss Pediatric | |
| | Expectorant; | |
| | Phenhist Expectorant; | |
| | Robafen DAC; | |
| | Robitussin-DAC; | |
| | Ryna-CX Liquid; | |
| | Tussar-2; | |
| | Tussar SF; | |
| 132 | Ambenyl-D | pseudoephedrine, dextromethorphan and |
| | Decongestant Cough | guaifenesin. |
| | Formula; | |
| | Anatuss DM; | |
| | Benylin Multi- | |
| | Symptom; | |
| | Concentrin; | |
| | Dimacol Caplets; | |
| | Dorcol Children's | |
| | Cough; | |
| | Novahistine DMX | |
| | Liquid; | |
| | PediaPressin Pediatric | |
| | Drops; | |
| | Primatuss Cough | |
| | Mixture 4D; | |
| | Rhinosyn-X; | |
| | Robitussin Cold and | |
| | Cough Liqui-Gels; | |
| | Ru-Tuss Expectorant; | |
| | Sudafed Children's | |
| | Non-Drowsy Cold & | |
| | Cough; | |
| | Sudafed Children's | |
| | Cold & Cough; | |
| 133 | Cophene-XP; | pseudoephedrine, hydrocodone and |
| | Detussin Expectorant; | guaifenesin. |
| | Duratuss HD; | |
| | Entuss-D Jr; | |
| | Med-Hist Exp; | |
| | SRC Expectorant; | |
| | Tussafin Expectorant; | |
| | Vanex Expectorant; | |
| 134 | Entuss-D; | pseudoephedrine, hydrocodone and potassium |
| | Protuss-D; | guaiacolsulfonate. |
| 135 | | A decongestant, an antitussive, an |
| | | expectorant and an analgesic. |
| 136 | | phenylpropanolamine, dextromethorphan, |
| | | guaifenesin and acetaminophen. |
| 137 | Comtrex Cough | pseudoephedrine, dextromethorphan, |
| | Formula; | guaifenesin and acetaminophen. |
| | Robitussin Cold, | |
| | Cough & Flu Liqui- | |
| | Gels; | |
| | Sudafed Cold & | |
| | Cough Liquid Caps; | |
| | Vicks DayQuil Multi- | |
| | Symptom Cold/Flu | |
| | LiquiDaps; | |
| | Vicks DayQuil Multi- | |
| | Symptom Cold/Flu | |
| | Relief; | |
| 138 | | A decongestant and an expectorant. |
| 139 | Broncholate; | ephedrine and guaifenesin. |
| 140 | KIE; | ephedrine and potassium iodide. |
| 141 | Deconsal Pediatric; | phenylephrine and guaifenesin. |
| | Endal; | |
| | Rescon-GG; | |
| | Sinupan; | |
| 142 | Ami-Tex; | phenylephrine, phenylpropanolamine and |
| | Banex Liquid; | guaifenesin. |
| | Contuss; | |
| | Despec; | |
| | Despec SF; | |
| | Dura-Gest; | |
| | Dura-Tex; | |
| | Enomine; | |
| | Entex; | |
| | Entex Liquid; | |
| | Norel; | |
| | Sil-Tex; | |
| 143 | Ami-Tex LA; | phenylpropanolamine and guaifenesin. |
| | Banex-LA; | |
| | Conex; | |
| | Despec-SR Caplets; | |
| | Dura-Vent; | |
| | Entex LA; | |
| | Exgest LA; | |
| | Guaifenex PPA 75; | |
| | Guaipax; | |
| | Myminic Expectorant; | |
| | Naldecon-EX | |
| | Children's Syrup; | |
| | Naldecon-EX Pediatric | |
| | Drops; | |
| | Partuss LA; | |
| | Phenylfenesin LA; | |
| | Profen II; | |
| | Profen-LA; | |
| | Prominic Expectorant; | |
| | Rymed-TR Caplets; | |
| | Silaminic Expectorant; | |
| | Sildecon-E Pediatric | |
| | Drops; | |
| | SINUvent; | |
| | Snaplets-EX; | |
| | Stamoist LA; | |
| | Triaminic Expectorant; | |
| | Triphenyl Expectorant; | |
| | ULR-LA; | |
| | Vicks DayQuil Simus | |
| | Pressure and | |
| | Congestion Relief | |
| | Caplets; | |
| 144 | Anatuss LA; | pseudoephedrine and guaifenesin. |
| | Congess JR; | |
| | Congess SR; | |
| | Congestac Caplets; | |
| | Deconsal II; | |
| | Duratuss; | |
| | Entex PSE; | |
| | Eudal-SR; | |
| | Expressin 400 | |
| | Caplets; | |
| | Glycofed; | |
| | GP-500; | |
| | Guaifed; | |
| | Guaifed-PD; | |
| | Guaifenex PSE 60; | |
| | Guaifenex PSE 120; | |
| | GuaiMAX-D; | |
| | Guaitab; | |
| | Guaivent; | |
| | Guaivent PD; | |
| | Guai-Vent/PSE; | |
| | GuaiCough PE; | |
| | Guaituss PE; | |
| | Humibid Guaifenesin | |
| | Plus; | |
| | losal II; | |
| | Nalex; | |
| | Nalex Jr; | |
| | Nasabid; | |
| | Nasatab LA; | |
| | PanMist-JR; | |
| | Respa-1^{st}; | |
| | Respaire-60 SR; | |
| | Respaire-120 SR; | |
| | Robitussin-PE; | |
| | Robitussin Severe | |
| | Congestion Liqui-Gels; | |
| | Ru-Tuss DE; | |
| | Rymed; | |
| | Rymed Liquid; | |
| | Sinufed Timecelles; | |
| | Sinutab Non-Drying | |
| | No Drowsiness Liquid | |
| | Caps; | |
| | Stamoist E; | |
| | Sudafed Non-Drowsy | |
| | Non-Drying Sinus | |
| | Liquid Caps; | |
| | Sudal 60/500; | |
| | Sudal 120/600; | |
| | Touro LA Caplets; | |
| | Tuss-LA; | |
| | V-Dec-M; | |
| | Versacaps; | |
| | Zephrex; | |
| | Zephrex-LA; | |
| 145 | | A decongestant, an expectorant and an |
| | | analgesic. |
| 146 | Fendol; | phenylephrine, guaifenesin, acetaminophen, |
| | | salicylamide and caffeine. |
| 147 | | An antihistamine and a decongestant |
| 148 | Alerid-D | cetirzine and pseudoephedrine |
| 149 | Claritin Reditabs | loratadine and pseudoephedrine |
| | Claritin 24-Hour | |
| | Claritin 12-Hour | |

In an embodiment of the present method, a subject in need of prevention, treatment or amelioration of a cold and/or a cough is treated by administering to the subject a cyclooxygenase-2 selective inhibitor or prodrug thereof and one or more colds and cough active ingredient. In one embodiment, the subject is treated with an amount of a colds and cough active ingredient and an amount of a Cox-2 selective inhibitor, where the amount of the colds and cough active ingredient and the amount of the Cox-2 selective inhibitor together provide a dosage or amount of the combination that is sufficient to constitute an effective amount of the combination. The effective amount can be a therapeutic amount, and it can be an amount that is an effective amount for the prevention, treatment or amelioration of a cold or a cough.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of each agent for use in the combination therapy which will achieve the goal of improvement in the severity of neurological or psychiatric disorder and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711. Furthermore, detailed prescribing information is available over the internet, or from the manufacturer or distributor, for each of the commercial colds and cough active ingredients that are described in Table 3.

In the present method, the amount of the colds and cough active ingredient that is used is such that, when administered with the cyclooxygenase-2 selective inhibitor, it is sufficient to constitute an effective amount of the combination. It is preferred that the dosage amount of the colds and cough active ingredient and the dosage amount of the cyclooxygenase-2 selective inhibitor constitute a therapeutically effective amount of the combination.

It is well known that different colds and cough active ingredients have different levels of potency and that recommended dosage levels vary considerably. The recommended dosage level for a commercial colds and cough active ingredient can be found in the prescribing information that is published by the distributor as described above.

The frequency of dose will depend upon the half-life of the colds and cough active ingredient molecule. If the colds and cough active ingredient has a short half life (e.g. from about 2 to 10 hours) it may be necessary to give one or more doses per day. Alternatively, if the colds and cough active ingredient has a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an ingredient taken once every two days would be expressed as a dosage rate of 50 mg/day. Similarly, the dosage rate of an ingredient where 50 mg is taken twice per day would be expressed as a dosage rate of 100 mg/day.

For the purposes of calculation of a dosage rate for the present method, the weight of an adult human is assumed to be 70 kg.

The amount of Cox-2 selective inhibitor that is used in the subject method may be an amount that, when administered with the colds and cough active ingredient, is sufficient to constitute an effective amount of the combination. Preferably, such amount would be sufficient to provide a therapeutically effective amount of the combination. The therapeutically effective amount can also be described herein as an amount that is effective for the prevention, treatment or amelioration of a cold and/or a cough.

In the present method, the amount of Cox-2 selective inhibitor that is used in the novel method of treatment preferably ranges from about 0.01 to about 100 milligrams per day per kilogram of body weight of the subject (mg/day·kg), more preferably from about 0.1 to about 50 mg/day·kg, even more preferably from about 1 to about 20 mg/day·kg.

When the Cox-2 selective inhibitor comprises rofecoxib, it is preferred that the amount used is within a range of from about 0.15 to about 1.0 mg/day·kg, and even more preferably from about 0.18 to about 0.4 mg/day·kg.

When the Cox-2 selective inhibitor comprises etoricoxib, it is preferred that the amount used is within a range of from about 0.5 to about 5 mg/day·kg, and even more preferably from about 0.8 to about 4 mg/day·kg.

When the Cox-2 selective inhibitor comprises celecoxib, it is preferred that the amount used is within a range of from about 1 to about 10 mg/day·kg, even more preferably from about 1.4 to about 8.6 mg/day·kg, and yet more preferably from about 2 to about 3 mg/day·kg.

When the Cox-2 selective inhibitor comprises parecoxib sodium, it is preferred that the amount used is within a range of from about 0.1 to about 3 mg/day·kg, and even more preferably from about 0.3 to about 1 mg/day·kg.

The combination of a colds and cough active ingredient and a Cox-2 selective inhibitor can be supplied in the form of a novel therapeutic composition that is believed to be within the scope of the present invention. The relative amounts of each component in the therapeutic composition may be varied and may be as described just above. The colds and cough active ingredient and Cox-2 selective inhibitor that are described above can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier, a pharmaceutical composition is formed. A pharmaceutical composition of the present invention is directed to a composition suitable for the prevention, treatment or amelioration of colds and/or coughs. The pharmaceutical composition comprises a pharmaceutically acceptable carrier, one or more colds and cough active ingredient, and a cyclooxygenase-2 selective inhibitor.

Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions, include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Notwithstanding the above description of certain alkali metal and alkali earth metal ions as being pharmaceutically acceptable cations, it should be recognized that it is preferred that the cold and cough active ingredient of the present invention be one that is free of an isolated metal salt of the cold and cough active ingredient. In other words, when the colds and cough active ingredient is one that can exist in a free acid form or in a metal salt form, it is preferred that at least some portion of the cold and cough active ingredient be present in its free acid form, rather than in an isolated metal salt form. It is more preferred that when the cold and cough active ingredient comprises an analgesic, the analgesic is free of an isolated metal salt of the analgesic. In other words, it is preferred that at least some portion of the analgesic be present in its free acid form, rather than its metal salt form. It is yet more preferred that when the cold and cough active ingredient comprises acetaminophen, the acetaminophen is free of an isolated metal salt of the acetaminophen. In other words, it is preferred that at least some portion of the acetaminophen be present in its free acid form, rather than its metal salt form.

In some cases, in particular where a subject is adversely affected by acetaminophen, it is preferred that the novel method and compositions be free of acetaminophen.

Also included in the combination of the invention are the isomeric forms and tautomers and the pharmaceutically-acceptable salts of antipsychotic agents and cyclooxygenase-2 selective inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, enanthic, decanoic and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method and compositions of the present invention are useful for, but not limited to, the prevention, inhibition, and amelioration of a cold and/or a cough in a subject that is need of such treatment. By way of example, the method and compositions would be useful for the prevention, treatment and amelioration of runny nose, nasal congestion, lung congestion, bronchial irritation, neuritis, neuralgia, sore throat, pain, aches, inflammation, sneezing, coughing, upper respiratory infections, allergic rhinitis, otitis, sinusitis, coryza, itchy and watery eyes, and the like, or any two or more of the symptoms described above.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of colds and/or cough, or the symptoms associated with, but not limited to those disorders. Besides being useful for human treatment, these combinations are also useful for treatment of mammals, including horses, dogs, cats, rats, mice, sheep, pigs, etc.

The term “subject” for purposes of treatment includes a subject who is in need of the prevention of, or who has a cold or a cough. The subject is typically an animal, and yet more typically is a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any animal subject, and preferably is a subject that is in need of prevention and/or treatment of a cold and/or a cough. The subject may be a human subject who is at risk for a cold or cough. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of a cyclooxygenase-2 inhibitor agent and a colds and cough active ingredient, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

Although the combination of the present invention may include administration of a colds and cough active ingredient component and a cyclooxygenase-2 selective inhibitor component within an effective time of each respective component, it is preferable to administer both respective components contemporaneously, and more preferable to administer both respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The present invention further comprises kits that are suitable for use in performing the methods of prevention, treatment, or inhibition described above. In one embodiment, the kit contains a first dosage form comprising one or more colds and cough active ingredient in one or more of the forms identified above and a second dosage form comprising one or more of the cyclooxygenase-2 selective inhibitors or prodrugs thereof identified above, in quantities sufficient to carry out the methods of the present invention. Preferably, the first dosage form and the second dosage form together comprise a therapeutically effective amount of the compounds for the treatment, prevention, or amelioration of a cold and/or a cough.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a Weight basis unless otherwise indicated.

### COMPARATIVE EXAMPLE 1

This example shows the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157⁰-159° C.; and a calculated composition of C₁₇H₁₄N₃ 02 SF₃; C, 53.54; H, 3.70; N, 11.02. The composition that was found by analysis was: C, 53.17; H, 3.81; N, 10.90.

### EXAMPLE 2

This illustrates the production of a composition containing celecoxib and the combination of an antihistamine, a decongestant, an antitussive and an analgesic, and of a pharmaceutical composition containing the combination.

The combination of an antihistamine, a decongestant, an antitussive and an analgesic may be supplied by any one of several commercially available preparations. One such preparation is ALKA-SELTZER® PLUS LIQUI-GELS COLD & COUGH MEDICINE, available from Bayer Corporation, Elkhart, Ind. Each liqui-gel capsule of ALKA-SELTZER® PLUS LIQUI-GELS COLD & COUGH MEDICINE contains chlorpheniramine maleate, 2 mg; pseudoephedrine hydrochloride, 30 mg; dextromethorphan hydrobromide, 10 mg; and acetaminophen, 325 mg.

Celecoxib can be prepared as described in Comparative Example 1, or it can be obtained under the trade name CELEBREX® from Pharmacia Corporation, Peapack, N.J.

A therapeutic composition of the present invention can be formed by intermixing chlorpheniramine maleate, 2 g; pseudoephedrine hydrochloride, 30 g; dextromethorphan hydrobromide, 10 g; acetaminophen, 325 g, and 4-[5-(4-methylphenyl)-3-(trifluoromethyl)-1H-pyrazol-1-yl]benzenesulfonamide (200 g, as produced in Comparative Example 1, or as available from Pharmacia Corporation, Peapack, N.J., under the tradename CELEBREX®), in a suspension or solution with a sterile pharmaceutically acceptable liquid. After mixing, the combination of chlorpheniramine maleate, pseudoephedrine hydrochloride, dextromethorphan hydrobromide, acetaminophen, and celecoxib forms a therapeutic composition that is sufficient for the production of about 1000 human single dose units. Each single dose unit contains about 2 mg of chlorpheniramine maleate, 30 mg of pseudoephedrine hydrochloride, 10 mg of dextromethorphan hydrobromide, 325 mg of acetaminophen and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule contains can contain about the same amount of the active ingredients as each of the single dose units of the liquid preparation described above.

Therapeutic and pharmaceutical compositions comprising a combination of any of the cyclooxygenase-2 selective inhibitors and any of the sources of cold and cough active ingredients that are described above can be formed by similar methods.

### EXAMPLE 3

This illustrates the production of a composition containing celecoxib and aciclovir, and of a pharmaceutical composition containing the combination.

Aciclovir (acyclovir) is available in the form of capsules, tablets and as a suspension under the trade name ZOVIRAX® from GlaxoSmith Kline, Research Triangle Park, N.C. Celecoxib can be prepared as described in Comparative Example 1, or it can be obtained under the trade name CELEBREX® from Pharmacia Corporation, Peapack, N.J.

A therapeutic composition of the present invention can be formed by intermixing solid or powdered aciclovir (400 g, available as ZOVIRAX®, from GlaxoSmithKline), and 4-[5-(4-methylphenyl)-3-(trifluoromethyl)-1H-pyrazol-1-yl]benzenesulfonamide (200 g, as produced in Comparative Example 1, or as available from Pharmacia Corporation, Peapack, N.J., under the tradename CELEBREX®), in a laboratory mill or mixing device suitable for intimate mixing of powders without substantial generation of shear or temperature sufficient to degrade either of the two compounds. After mixing, the combination of aciclovir and celecoxib forms a therapeutic composition that is sufficient for the production of about 1000 human single dose units. Each single dose unit contains about 400 mg of aciclovir and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule contains 400 mg of aciclovir and 200 mg celecoxib.

Alternatively, the aciclovir (preferably in the form of a suspension) and the celecoxib may be dissolved or suspended into a liquid carrier, such as, for example, normal saline solution, to form a pharmaceutical composition suitable for human consumption. A single dosage of the liquid pharmaceutical composition for human use would be a volume sufficient to provide 400 mg of aciclovir and 200 mg of celecoxib.

Therapeutic and pharmaceutical compositions comprising a combination of any of the cyclooxygenase-2 selective inhibitors and any of the colds and cough active ingredients that are described above can be formed by similar methods.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method for the treatment, prevention and amelioration of colds and/or cough in a subject in need of such treatment, prevention and amelioration, the method comprising administering to the subject a cyclooxygenase-2 selective inhibitor or prodrug thereof and one or more colds and cough active ingredient.

2. The method according to claim 1, except that when the colds and cough active ingredient is an analgesic, it is free of the isolated salt form of acetaminophen.

3. The method according to claim 1, wherein at least a portion of the colds and cough active ingredient is free of an isolated metal salt form of the colds and cough active ingredient.

4. The method according to claim 1, wherein the cyclooxygenase-2 selective inhibitor and a colds and cough active ingredient are administered to the subject in combination and where the amount of the cyclooxygenase-2 selective inhibitor and the amount of the one or more colds and cough active ingredient together comprise an effective amount of the combination.

5. The method according to claim 4, wherein the effective amount of the combination is a therapeutically effective amount for the treatment, prevention and/or amelioration of colds and cough in the subject.

6. The method according to claim 1, wherein the colds and cough active ingredient comprises an antihistamine, antitussive, analgesic, expectorant, decongestant, anticholinergic, antiviral agent, or a mixture of two or more thereof.

7. The method according to claim 6, wherein the colds and cough active ingredient comprises an antihistamine.

8. The method according to claim 7, wherein the antihistamine is selected from the group consisting of azatadine, bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, promethazine, pyrilamine, triprolidine, cetirzine, loratadine, and mixtures thereof.

9. The method according to claim 6, wherein the colds and cough active ingredient comprises an antitussive.

10. The method according to claim 9, wherein the antitussive is selected from the group consisting of codeine, dihydrocodeine, hydrocodone, hydrocodone bitartrate, hydromorphone, carbetapentane, caraminphen, dextromethorphan, chlorphedianol, noscarpine, and mixtures thereof.

11. The method according to claim 6, wherein the colds and cough active ingredient comprises an analgesic.

12. The method according to claim 11, wherein the analgesic is selected from the group consisting of acetaminophen, aspirin, salicylamide, sodium salicylate, indomethacin, ibuprofen, naproxen, flubiprofen, carprofen, tiaprofenic acid, cicloprofen, detoprofen, ketorolac, etodolac, and mixtures thereof.

13. The method according to claim 6, wherein the colds and cough active ingredient comprises an expectorant.

14. The method according to claim 13, wherein the expectorant is selected from the group consisting of guaifenesin, glycerol guaiacolate, terpin hydrate, ammonium chloride, N-acetylesteine, bromhexine, ambroxol, domiodol, 3-iodo-1,2-propanediol, and mixtures thereof.

15. The method according to claim 6, wherein the colds and cough active ingredient comprises a decongestant.

16. The method according to claim 15, wherein the decongestant is selected from the group consisting of ephedrine, ephinephrine, levodesoxyephedrine, oxymetazoline, naphazoline, phenylephrine, phenylpropanolamine, propylhexedrine, pseudoephedrine, xylometazoline, and mixtures thereof.

17. The method according to claim 6, wherein the colds and cough active ingredient comprises an anticholinergic.

18. The method according to claim 17, wherein the anticholinergic comprises homatropine.

19. The method according to claim 6, wherein the colds and cough active ingredient comprises an antiviral agent.

20. The method according to claim 19, wherein the antiviral agent is selected from the group consisting of dipyridamole, ICI 130,685, impulsin, pleconaril, zanamivir, oseltamivir, famciclovir, valaciclovir, valganciclovir, aciclovir, ganciclovir, idoxuridine, vidarabine, trifluridine, penciclovir, valacyclovir, foscarnet, ribavarin, amantadine, rimantadine, cidofovir, and mixtures of two or more of these compounds.

21. The method according to claim 6, wherein the colds and cough active ingredient is selected from the group consisting of azatadine, bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, promethazine, pyrilamine, triprolidine, cetirzine, loratadine, codeine, dihydrocodeine, hydrocodone, hydrocodone bitartrate, hydromorphone, carbetapentane, caraminphen, dextromethorphan, acetaminophen, aspirin, salicylamide, sodium salicylate, guaifenesin, ephedrine, ephinephrine, levodesoxyephedrine, oxymetazoline, naphazoline, phenylephrine, phenylpropanolamine, propylhexedrine, pseudoephedrine, xylometazoline, homatropine, dipyridamole, ICI 130,685, impulsin, pleconaril, zanamivir, oseltamivir, famciclovir, valaciclovir, valganciclovir, aciclovir, ganciclovir, idoxuridine, vidarabine, trifluridine, penciclovir, valacyclovir, foscarnet, ribavarin, amantadine, rimantadine, cidofovir, and mixtures of two or more thereof.

22. The method according to claim 1, wherein the cyclooxygenase-2 selective inhibitor or prodrug thereof has a cyclooxygenase-2 IC₅₀ of less than about 0.2 μmol/L.

23. The method according to claim 22, wherein the cyclooxygenase-2 selective inhibitor or prodrug thereof has a cyclooxygenase-1 IC₅₀ of at least about 1 μmol/L.

24. The method according to claim 23, wherein the cyclooxygenase-2 selective inhibitor or prodrug thereof has a cyclooxygenase-1 IC₅₀ of at least about 10 μmol/L.

25. The method according to claim 1, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, and NS-398.

26. The method according to claim 25, wherein the cycloxygenase-2 selective inhibitor comprises a compound selected from the group consisting of celecoxib, valdecoxib and parecoxib.

27. The method according to claim 6, wherein the one or more colds and cough active ingredients comprise an antihistamine and an antitussive.

28. The method according to claim 6, wherein the one or more colds and cough active ingredient comprises an antihistamine, an antitussive, and an analgesic.

29. The method according to claim 6, wherein the one or more colds and cough active ingredient comprises an antihistamine, an antitussive, and an expectorant.

30. The method according to claim 6, wherein the one or more colds and cough active ingredient comprises an antihistamine, a decongestant and an antitussive.

31. The method according to claim 6, wherein the one or more colds and cough active ingredient comprises an antihistamine, a decongestant, an antitussive and an analgesic.

32. The method according to claim 6, wherein the one or more colds and cough active ingredient comprises an antihistamine, a decongestant, an antitussive and an expectorant.

33. The method according to claim 6, wherein the one or more colds and cough active ingredient comprises an antihistamine, a decongestant, an antitussive, an expectorant and an analgesic.

34. The method according to claim 6, wherein the one or more colds and cough active ingredient comprises an antihistamine, a decongestant and an expectorant.

35. The method according to claim 6, wherein the one or more colds and cough active ingredient comprises an antihistamine, a decongestant, an expectorant and an analgesic.

36. The method according to claim 6, wherein the one or more colds and cough active ingredient comprises an antihistamine and an expectorant.

37. The method according to claim 6, wherein the one or more colds and cough active ingredient comprises an antitussive and an analgesic.

38. The method according to claim 6, wherein the one or more colds and cough active ingredient comprises an antitussive and an antichlolinergic.

39. The method according to claim 6, wherein the one or more colds and cough active ingredient comprises an antitussive and an expectorant.

40. The method according to claim 6, wherein the one or more colds and cough active ingredient comprises a decongestant and an antitussive.

41. The method according to claim 6, wherein the one or more colds and cough active ingredient comprises a decongestant, an antitussive and an analgesic.

42. The method according to claim 6, wherein the one or more colds and cough active ingredient comprises a decongestant, an antitussive and an expectorant.

43. The method according to claim 6, wherein the one or more colds and cough active ingredient comprises a decongestant, an antitussive, an expectorant and an analgesic.

44. The method according to claim 6, wherein the one or more colds and cough active ingredient comprises a decongestant and an expectorant.

45. The method according to claim 6, wherein the colds and cough active ingredient comprises an antihistamine and a decongestant.

46. A composition for the treatment, prevention and amelioration of colds and/or cough in a subject in need of such treatment, prevention and amelioration, the composition comprising a cyclooxygenase-2 selective inhibitor and a colds and cough active ingredient.

47. The composition according to claim 46, except that when the colds and cough active ingredient is an analgesic, it is free of the isolated salt form of acetaminophen.

48. The composition according to claim 46, wherein at least a portion of the colds and cough active ingredient is free of an isolated metal salt form of the colds and cough active ingredient.

49. The composition according to claim 46, wherein the colds and cough active ingredient is selected from the group consisting of azatadine, bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, promethazine, pyrilamine, triprolidine, cetirzine, loratadine, codeine, dihydrocodeine, hydrocodone, hydrocodone bitartrate, hydromorphone, carbetapentane, caraminphen, dextromethorphan, acetaminophen, aspirin, salicylamide, sodium salicylate, guaifenesin, ephedrine, ephinephrine, levodesoxyephedrine, oxymetazoline, naphazoline, phenylephrine, phenylpropanolamine, propylhexedrine, pseudoephedrine, xylometazoline, homatropine, dipyridamole, ICI 130,685, impulsin, pleconaril, zanamivir, oseltamivir, famciclovir, valaciclovir, valganciclovir, aciclovir, ganciclovir, idoxuridine, vidarabine, trifluridine, penciclovir, valacyclovir, foscarnet, ribavarin, amantadine, rimantadine, cidofovir, and mixtures of two or more thereof.

50. The composition according to claim 46, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, NS-398, mixtures of any two or more thereof, and prodrugs thereof.

51. A composition for the treatment, prevention and amelioration of colds and/or cough in a subject in need of such treatment, prevention and amelioration, the composition comprising a cyclooxygenase-2 selective inhibitor selected from the group consisting of celecoxib, parecoxib and valdecoxib, and a colds and cough active ingredient selected from the group consisting of chlorpheniramihe, cetirzine, loratadine, codeine, hydrocodone, carbetapentane, dextromethorphan, aspirin, guaifenesin, ephedrine, ephinephrine, phenylephrine, phenylpropanolamine, pseudoephedrine, impulsin, pleconaril, aciclovir, and ganciclovir.

52. A composition for the treatment, prevention and amelioration of colds and/or cough in a subject in need of such treatment, prevention and amelioration, the composition comprising a cyclooxygenase-2 selective inhibitor and a combination of two or more colds and cough active ingredients.

53. The composition according to claim 52, wherein the combination of two or more colds and cough active ingredients comprises at least two agents selected from the group consisting of antihistamine, antitussive, analgesic, expectorant, decongestant, anticholinergic, and antiviral agent.

54. The composition according to claim 53, wherein the combination of two or more colds and cough active ingredients comprises at least two agents selected from the group consisting of azatadine, bromodiphenhydramine, brompheniramine, brompheniramine maleate, carbinoxamine, chlorpheniramine, dexchlorpheniramine, diphenhydramine, doxylamine, phenindamine, pheniramine, phenyltoloxamine, promethazine, pyrilamine, triprolidine, cetirzine, loratadine, codeine, dihydrocodeine, hydrocodone, hydrocodone bitartrate, hydromorphone, carbetapentane, caraminphen, dextromethorphan, aspirin, salicylamide, sodium salicylate, guaifenesin, ephedrine, ephinephrine, levodesoxyephedrine, oxymetazoline, naphazoline, phenylephrine, phenylpropanolamine, propylhexedrine, pseudoephedrine, xylometazoline, homatropine, dipyridamole, ICI 130,685, impulsin, pleconaril, zanamivir, oseltamivir, famciclovir, valaciclovir, valganciclovir, aciclovir, ganciclovir, idoxuridine, vidarabine, trifluridine, penciclovir, valacyclovir, foscarnet, ribavarin, amantadine, rimantadine, cidofovir, and mixtures of two or more thereof.

55. The composition according to claim 54, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, NS-398, mixtures of any two or more thereof, and prodrugs thereof.

56. A pharmaceutical composition for the treatment, prevention and amelioration of colds and/or cough in a subject in need of such treatment, prevention and amelioration, the composition comprising a cyclooxygenase-2 selective inhibitor, a colds and cough active ingredient, and a pharmaceutically-acceptable excipient.

57. A kit that is suitable for use in the treatment, prevention or amelioration of colds and/or cough, the kit comprises a first dosage form comprising a colds and cough active ingredient and a second dosage form comprising a cyclooxygenase-2 selective inhibitor or prodrug thereof, in quantities which comprise a therapeutically effective amount of the combination of the compounds for the treatment, prevention, or amelioration of colds and/or cough.
